SEQUENCE LISTING (5 sequences)

SEQ ID NO: 1 - protein, 10 aa, synthetic construct
  source 1..10: note = Synthetic peptide used for enrichment studies
YILAGVENSK

SEQ ID NO: 2 - protein, 14 aa, synthetic construct
  source 1..14: note = Synthetic peptide used for enrichment studies
GAGSSEPVTGLDAK

SEQ ID NO: 3 - protein, 12 aa, synthetic construct
  source 1..12: note = Synthetic peptide used for enrichment studies
TPVISGGPYEYR

SEQ ID NO: 4 - protein, 12 aa, synthetic construct
  source 1..12: note = Synthetic peptide used for enrichment studies
TPVITGAPYEYR

SEQ ID NO: 5 - protein, 13 aa, synthetic construct
  source 1..13: note = Synthetic peptide used for enrichment studies
DGLDAASYYAPVR

(19) United States
(12) Patent Application Publication
(10) Pub. No.: US 2026/0266827 A1
(43) Pub. Date: Sep. 10, 2026

(54) COMPOSITIONS, METHODS AND KITS FOR PROFILING PROTEIN MODIFICATIONS

(71) Applicant: Purdue Research Foundation, West Lafayette, IN (US)

(72) Inventors: Weiguo Tao, West Lafayette, IN (US); Jie Sun, West Lafayette, IN (US)

(21) Appl. No.: 19/162,929

(22) PCT Filed: Mar. 6, 2024
(86) PCT No.: PCT/US2024/018669

§ 371 Date: Sep. 6, 2025

Related U.S. Application Data
(60) Provisional application No. 63/488,759, filed on Mar. 6, 2023.

(51) Int. Cl. G01N 33/575; G01N 33/543

(52) U.S. Cl. CPC G01N 33/57585; G01N 33/54346; G01N 2440/14

(57) ABSTRACT

Compositions of matter, methods of use and automation of processes for the characterization of biological samples are provided, such compositions and methods useful for monitoring the biological processes regulated through dynamic protein phosphorylation.

### PRIORITY

This patent application is related to and claims the priority benefit of U.S. Provisional Patent Application No. 63/488,759 filed Mar. 6, 2023. The content of the foregoing application is hereby incorporated by reference in its entirety into this disclosure.

### TECHNICAL FIELD

The present invention generally relates to compositions of matter, materials and methods for assaying, profiling, and monitoring protein modifications. The present invention provides for monitoring of biological samples which can be of small volume. Also described herein are the use of the compositions and methods in apparatus for assaying, profiling, and monitoring protein modifications, which can be automated to varying degrees.

### SEQUENCE LISTINGS

The sequences herein (SEQ ID NOS: 1-5) are also provided in computer readable form encoded in a file filed herewith and incorporated herein by reference. The information recorded in computer readable form is identical to the written Sequence Listings provided below, pursuant to 37 C.F.R. § 1.821(f).

LENGTHY TABLES
The patent application contains a lengthy table section. A copy of the table is available in electronic form from the USPTO web site (https://seqdata.uspto.gov/docdetail?docId=US20260266827A1). An electronic copy of the table will also be available from the USPTO upon request and payment of the fee set forth in 37 CFR 1.19(b)(3).

### BACKGROUND

This section introduces aspects that may help facilitate a better understanding of the disclosure. Accordingly, these statements are to be read in this light and are not to be understood as admissions about what is or is not prior art.

Many biological processes are regulated through dynamic protein phosphorylation. Reversible protein phosphorylation has been shown to be among the most widespread of all known post-translational modifications; for example, it is estimated that about 30% of all human proteins are phosphorylated at one time or another. This post-translational modification plays an important role in the regulation of many cellular functions, including growth, differentiation, and signaling. Changes in phosphorylation dynamics within the cell has been linked to the onset and development of numerous diseases, for example some forms of cancer. While monitoring disease-relevant phosphorylation events in circulating biofluids is highly appealing, it has proven to be technically challenging using conventional techniques.

Protein phosphorylation regulates almost every essential cellular function, and the state of phosphorylation events reveals the critical information about a disease status, highlighting that phosphoproteins have remarkable potential as biomarkers for disease diagnosis and prognosis. In recent decades, significant progress has been made to profile cell-wide phosphorylation events in disease-derived cell and animal models, and in diseased tissues. By directly quantifying changes in global phosphorylation in response to environmental perturbation, stimulation, or intervention, mass spectrometry (MS)-based proteomics enables insightful studies into regulatory pathways and network closely associated with cellular function and disease progression. More recently, attempts have been made to measure phosphoproteins in biofluids for biomarker screening, disease progression and therapeutic assessment. Compared with tissue biopsy, liquid biopsy is more universal, less heterogeneous, easier specimen acquisition and processing, and suitable for longitudinal disease monitoring. However, monitoring phosphoprotein changes in biofluids is extremely challenging, due to their labile nature in the presence of phosphatases and their undetectable concentration in comparison to the extremely abundant proteins in biofluid.

Extracellular vesicles (EVs) have emerged as intriguing surrogates for intercellular communication and biological sources for diagnosis and therapeutics. Our previous work demonstrated that the bilayer membrane structure of EVs effectively protect proteins, nucleic acids and other cargo molecules from circulating phosphatases and proteases and the isolation of EVs from biofluids can alternatively reduce the complexity of biological samples by circumventing highly abundant proteins in biofluids, thus opening up a new research avenue for phosphoproteomics analysis. However, EV phosphoproteomics is greatly limited by relatively low EV isolation efficiency from highly complex clinically relevant samples such as plasma, the time-consuming steps for multiple desalting and enrichment of phosphopeptides accompanied by specimen loss during the process, and the fact that conventional techniques consume relatively large amount of clinical samples.

### BRIEF SUMMARY

The present invention generally relates to methods for assaying, profiling, and monitoring protein modifications and compositions of matter for doing so.

Also described herein are apparatus utilizing the methods and compositions for assaying, profiling, and monitoring protein modifications of the present invention. A further aspect of the present invention is that the materials and methods allow for the assay of small volume samples. A further aspect of the present invention are discretely packaged assay components which are suitable for use in partially automated or fully automated biological assay apparatus or otherwise mechanized autonomous or semi-autonomous device.

Introduced here is a functionally tunable material and a strategy, extracellular vesicles to phosphoproteins (EVTOP), which achieves one-pot EV isolation, extraction and digestion of EV proteins, and enrichment of phosphopeptides starting with only trace amount of biofluids. EVs are efficiently isolated by magnetic beads functionalized with Ti(IV) ions and a membrane-penetrating peptide, octa-arginine R8+, which also provide the hydrophilic surface to retain EV proteins during lysis. Subsequent on-bead digestion concurrently converts EVTOP to Ti(IV) ion-only surface for efficient enrichment of phosphopeptides for phosphoproteomic analyses. The streamlined, ultra-sensitive platform enabled us to quantify 500 unique EV phosphopeptides with only a few μL of plasma and over 1,200 phosphopeptides with 100 μL of cerebrospinal fluid (CSF). We demonstrated its clinical application of evaluating the outcome of chemotherapy of primary central nervous system lymphoma (PCNSL) patients with small volume of CSF, presenting a powerful tool for broad clinical applications.

In certain embodiments, a composition for recovery of phosphopeptides from cells and EVs is provided. Such a composition can comprise a solid phase support; at least a cleavable membrane binding/penetrating group functionalizing a surface of the solid phase support; and at least one metal ion or metal oxide functionalizing the surface of the solid support. The cleavable membrane binding/penetrating group can have an affinity for a cell or an EV. The metal ion or metal oxide can have an affinity for at least one phosphorylated residue in a phosphopeptide.

The metal ion of the composition can be selected from the group consisting of iron, copper, gallium, cobalt, nickel, calcium, zinc, cadmium, silver, palladium, platinum, and ruthenium. The metal ion can be selected from the group consisting of titanium, zirconium, aluminum, vanadium, lead, manganese and tin. The at least one metal ion or metal oxide can comprise a Ti(IV) ion. In certain embodiments, the at least one metal ion or metal oxide is positively charged.

The metal oxide can be selected from the group consisting of titanium oxide, zirconium oxide and tin oxide, aluminum oxide, vanadium oxide, lead oxide and manganese oxide.

The solid phase support can comprise a silica bead, magnetic bead, a polymer resin, or a combination of any two or more of the foregoing. In any of the foregoing described embodiments, the solid phase support can comprise nanoparticles, for example. In certain embodiments, the solid phase support comprises nanoparticles.

The composition can further comprise a silica and/or a polymer shell coating at least a portion of the surface of the solid phase support. The solid phase support can further comprise polyethylene glycol and a silica shell coating at least a portion of the surface of the solid phase support.

In certain embodiments, a membrane binding/penetrating group of the composition can be cleaved (e.g., from the solid phase support) by an enzyme, a chemical, by light, or by a combination of any two or more of the foregoing. The binding/penetrating group can be selected from the group consisting of cell/membrane-penetrating peptides, membrane analogs, and membrane-bound molecules. The binding/penetrating group can comprise at least octa-arginine R⁺₈.

The at least one metal ion or metal oxide can comprise a Ti(IV) ion, and the binding/penetrating group comprises at least octa-arginine R⁺₈.

In certain embodiments, when subjected to an enzyme, a chemical, light, or a combination of any two or more of the foregoing, the binding/penetrating group is cleaved from the solid phase support while the at least one metal ion or metal oxide remains attached to the surface of the solid phase support.

The EVs can comprise one or more exosomes, small EVs (sEVs), ectosomes, microparticles, and microvesicles.

Kits for identifying phosphorylated peptides or the lack thereof (e.g., in a biological fluid sample) are also provided. Such a kit can comprise, for example, a solid phase support; at least a cleavable membrane binding/penetrating group functionalizing a surface of the solid phase support (e.g., where the cleavable membrane binding/penetrating group having an affinity for a cell or an EV); and at least one metal ion or metal oxide functionalizing the surface of the solid support. There, the metal ion or metal oxide can have an affinity for at least one phosphorylated residue in a phosphopeptide. The solid phase support of the kit can comprise a silica bead, magnetic bead, a polymer resin, or a combination of any two or more of the foregoing. The metal ion (of the kit) can be selected from the group consisting of iron, copper, gallium, cobalt, nickel, calcium, zinc, cadmium, silver, palladium, platinum, and ruthenium. In certain embodiments of the kit, the metal ion is selected from the group consisting of titanium, zirconium, aluminum, vanadium, lead, manganese and tin. In certain embodiments of the kit, the at least one metal ion or metal oxide comprises a Ti(IV) ion. In certain embodiments of the kit, the at least one metal ion or metal oxide is positively charged. In certain embodiments of the kit, the metal oxide is selected from the group consisting of titanium oxide, zirconium oxide and tin oxide, aluminum oxide, vanadium oxide, lead oxide and manganese oxide. In certain embodiments of the kit, the solid phase support comprises nanoparticles. In certain embodiments of the kit, the solid phase support comprises nanoparticles.

The composition in the kit can further comprise a silica shell coating at least a portion of the surface of the solid phase support. The solid phase support (i.e., of the kit) can further comprises polyethylene glycol and a silica shell coating at least a portion of the surface of the solid phase support. In certain embodiments of the kit, a membrane binding/penetrating group of the composition is formulated such that it can be cleaved by an enzyme, a chemical, by light, or by a combination of any two or more of the foregoing. In certain embodiments of the kit, the binding/penetrating group is selected from the group consisting of cell/membrane-penetrating peptides, membrane analogs, and membrane-bound molecules. In certain embodiments of the kit, the binding/penetrating group comprises at least octa-arginine R⁺₈. In certain embodiments of the kit, the at least one metal ion or metal oxide comprises a Ti(IV) ion, and the binding/penetrating group comprises at least octa-arginine R⁺₈.

In certain embodiments of the kit, when subjected to an enzyme, a chemical, light, or a combination of any two or more of the foregoing, the binding/penetrating group can be cleaved from the solid phase support of the composition while the at least one metal ion or metal oxide remains attached to the surface of the solid phase support. In certain embodiments of the kit, the EVs comprise one or more of exosomes, sEVs, ectosomes, microparticles, and microvesicles.

Methods are also provided. For example, a method for one-pot capturing and enriching phosphoproteins, phosphopeptides, or both from a biofluid sample is provided. Such a method can comprise: capturing EVs from a biofluid sample taken from a subject using a composition hereof; and enriching the captured EVs by: lysing the captured EVs while captured on the solid phase support, digesting the captured EVs to generate phosphopeptides, and cleaving the at least one membrane binding/penetrating group from the solid phase support.

The lysing step of the method can be performed through application of a reagent in an amount sufficient to lyse the captured EV. The reagent can comprise lithiumdodecyl sulfate.

The digesting the captured EVs step of the method can comprise digesting lysed proteins with Lys-C, trypsin, and/or other enzymes.

In certain embodiments of the method, digesting the captured EVs is performed for a time period and at a predefined temperature.

The method can further comprise quantifying the phosphopeptides to determine if the subject is experiencing an upregulation, downregulation, or normal expression of the phosphopeptides as compared to a control sample, wherein upregulation or downregulation is indicative of the subject experiencing an active disease state and normal expression is indicative of the subject not experiencing the active disease state.

Additionally or alternatively, the method can further comprise administering a treatment to the subject before or after collecting the biofluid sample from the subject. The treatment can comprise chemotherapy, for example.

In certain embodiments, the method further comprises quantifying the phosphopeptides to determine if the subject is experiencing an upregulation, downregulation, or normal expression of the phosphopeptides as compared to a control sample, wherein upregulation or downregulation is indicative of the subject experiencing an active disease state and normal expression is indicative of the subject not experiencing the active disease state. The disease state can be cancer, for example.

In certain embodiments, the phosphopeptides comprise one or more of the following: SPP1, Pro-thyrotropin-releasing hormone (TRH), Tenascin-C(TNC), Selenoprotein P (SEPP1), Fibrinogen beta chain (FGB/FIBB), Laminin subunit beta-1 (LAMB1), TNX, Histidine-rich glycoprotein (HRG), Golgi-associated kinase 1B (GAK1B) and Insulin-like growth factor-binding protein 5 (IGFBP5/IBP5).

The method can further comprise administering a treatment to the subject before collecting the biofluid sample from the subject; and quantifying the phosphopeptides to determine if the subject is experiencing an upregulation, downregulation, or normal expression of the phosphopeptides as compared to a control sample, wherein upregulation or downregulation is indicative of the subject experiencing an active disease state and normal expression is indicative of the subject not experiencing the active disease state. In such embodiments, the phosphopeptides can comprise one or more of the following: SPP1, Pro-thyrotropin-releasing hormone (TRH), Tenascin-C(TNC), Selenoprotein P (SEPP1), Fibrinogen beta chain (FGB/FIBB), Laminin subunit beta-1 (LAMB1), TNX, Histidine-rich glycoprotein (HRG), Golgi-associated kinase 1B (GAK1B) and Insulin-like growth factor-binding protein 5 (IGFBP5/IBP5).

Additionally, the method can further comprise collecting a first biofluid sample from the subject prior administering the treatment and a second biofluid sample from the subject after administering the treatment; wherein downregulation of the phosphopeptides in the second biofluid sample as compared to the first fluid sample is indicative of the treatment effectively treating the disease state. The biofluid sample can be, for example, plasma, urine, saliva, or cerebral spinal fluid (CSF). In certain embodiments, the biofluid sample is CSF. In certain embodiments, the biofluid sample comprises between about 10 μL to about 200 μL, inclusive of the end points. In certain embodiments, the biofluid sample comprises about 10 μL of plasma. In certain embodiments, the biofluid sample comprises about 100 μL of CSF. In certain embodiments, the biofluid sample comprises about 200 μL of urine. In certain embodiments, the biofluid sample comprises about 50 μL of saliva.

Uses of the composition hereof are also provided. In certain embodiments, uses of the composition for assessing a physiological response in a subject, or lack thereof, to an administered treatment is provided. The treatment can be chemotherapy, for example.

These and other features, aspects and advantages of the present invention will become better understood with reference to the following drawings, descriptions and claims.

### BRIEF DESCRIPTION OF THE DRAWINGS

The above and other objects, features, and advantages of the present invention will become more apparent when taken in conjunction with the following description and drawings, wherein:

FIGS. 1A-1G show data related to the characterization of magnetic bead (MB) fabrication and extracellular vesicle (EV) isolation, in particular with respect to the EV to phosphoprotein (EVTOP) MBs hereof. FIG. 1A shows a transmission electron microscopy (TEM) image of prepared MBs, FIG. 1B shows a TEM image of prepared silica shell coated MBs (MBs@SiO₂), FIG. 1C shows cerebrospinal fluid (CSF) EVs captured on EVTOP beads, FIG. 1D shows eluted CSF EVs, FIG. 1E shows a zeta potential characterization of the EVTOP preparation step by step, FIG. 1F shows a western blotting (WB) result of CSF EVs isolated by EVTOP, R⁺₈ only (bar values, for Flotillin 0.685, for CD9 0.705, and for β-actin 0.670), Ti(IV) only (bar values, for Flotillin 0.592, for CD9 0.534, and for β-actin 0.766), and ultracentrifugation methods (bar values, for Flotillin 0.261, for CD9 0.076, and for β-actin 0.241), and FIG. 1G shows a size distribution analysis of CSF EVs insolated by ultracentrifugation (concentration: 2.57×10⁸ particles/mL; average size: 161.4 nm) and the EVTOP method (concentration: 1.61×10⁹ particles/mL; average size: 125.6 nm).

FIGS. 2A-2G show data related to the effect of trypsin treatment of EVTOP beads on the capture efficiency of EVs. FIG. 2A shows a microscope image of CSF EVs isolated using EVTOP MBs (EVs were stained by DiD, including the image obtained by dark field, image obtained by bright field, and the merged image of them), FIG. 2B shows cleaved EVTOP MBs following trypsin digestion, FIG. 2C shows R⁺₈ only MBs, and FIG. 2D shows cleaved R⁺₈ only beads following trypsin digestion. FIG. 2E shows silica coated MBs. FIG. 2F is a zeta potential characterization of EVTOP MBs with/without trypsin digestion, FIG. 2G is a WB characterization of EVs isolated by EVTOP MBs with and without trypsin digestion, and FIG. 2H shows liquid chromatography with tandem mass spectrometry (LC-MS/MS) results of EV phosphoproteins directly isolated by EVTOP MBs both with and without trypsin digestion.

FIGS. 3A-3D show data related to the analysis of clinical CSF sample(s), with FIG. 3A showing a schematic of the steps for obtaining label free quantitation results related to identified phosphoproteins in control groups and primary central nervous system lymphoma (PCNSL) patient groups, such steps involving CSF EV Isolation (obtaining samples from both a healthy cohort (n=21) and a PCNSL cohort (n=11) and using phosphoproteomics to screen between healthy and PCNSL patients, and obtaining samples from a PCNSL cohort (n=11) to both pre-chemotherapy and post-chemotherapy monitor effects of treatment); Phosphopeptides Enrichment (performing EV phosphoproteomics on the collected samples); and Analysis (identifying targets from the enriched phosphopeptides, and analyzing via prm-PASEF for biomarkers of interest), FIG. 3B showing a heatmap and FIG. 3C showing a volcano plot of the differential expression of phosphoproteins, and FIG. 3D showing Gene Ontology (GO) and Kyoto Encyclopedia of Genes and Genomes (KEGG) pathway analyses data related to the upregulated phosphoproteins in the following patient groups (listed top to bottom): 1=secretory granule; 2=response to hypoxia; 3=Prion diseases; 4=positive regulation . . . production; 5=positive regulation phosphorylation; 6=PI3K-Akt signaling pathway; 7=peripheral nervous system regeneration; 8=neuropeptide hormone activity; 9=neuronal cell body; 10=neuron projection regeneration; 11=negative regulation . . . via . . . receptors; 12=negative regulation . . . pathway; 13=negative regulation of neuron death; 14=innate immune response; 15=hyaluronic acid binding; 16=Golgi apparatus; 17=Focal adhesion; 18=extracellular vesicle; 19=extracellular exosome; 20=endopeptidase inhibitor activity; 21=ECM-receptor interaction; 22=early endosome; 23=chemotaxis; 24=central nervous system development; 25=cell surface; 26=Cell adhesion molecules (CAMs).

FIG. 4A shows data from a capability evaluation of different monitored phosphoproteins from both CSF samples taken from a pre-chemotherapeutic cohort and from a post-chemotherapeutic cohort, with a zoomed view of selected phosphoproteins on the right, phosphoproteins identified from left to right as follows: 1=shadowMin; 2=IBP3; 3=IBP6; 4=FSTL3; 5=PRG4; 6=GLCE; 7=FBN1; 8=shadowMean; 9=ALBU; 10=K2C1; 11=KNG1; 12=FETUA; 13=CLUS; 14=FILA; 15=SPRL1; 16=A4; 17=GALA; 18=SP0N1; 19=NUCB1; 20=FINC; 21=TTIH2; 22=SCG1; 23=TICN3; 24=CYTC; 25=SULF2; 26=PCOC1; 27=FIBA; 28=LTBP1; 29=SODE; 30=BMP6; 31=EMAL4; 32=MARCS; 33=AMD; 34=MENT; 35=FBLN1; 36=TICN2; 37=SCG2; 38=CSPG2; 39=NCAN; 40=ECM2; 41=HEP2; 42=shadowMax; 43=IBP7; 44=SPP1; 45=TRH; 46=GAK1B; 47=TENA; 48=FIBB; 49=COLI; 50=IBP5; 51=SEPP1; 52=LAMB1; 53=HRG; 54=TENX.

FIG. 4B shows data related to patient expression of certain phosphoproteins post-chemotherapy, quantified from CSF samples and compared against pre-chemotherapy samples, the data indicating certain phosphoproteins showing reduced intensity (i.e., expression) following treatment.

FIG. 4C shows data relating to the intensity changes of different phosphoproteins selected by both machine learning and correlated patient analysis, including SPP1, TRH, TNC, SEPP2, IBP5 and TNX in both pre-chemotherapeutic samples and post-chemotherapeutic samples.

FIG. 5 shows a scheme for isolation of EVs from trace amount of biofluids and in situ attachment of the phosphopeptides for in-depth phosphoproteome profiling by EVTOP.

FIG. 6A shows data relating to the phosphoproteomics of EVs isolated by EVTOP beads from small amount of biofluids.

FIG. 6B shows data related to the identification of unique phosphopeptides and phosphoproteins from EVs isolated from different volumes of CSF samples using EVTOP beads and the ultracentrifugation method described herein (a=phosphopeptides isolated via ultracentrifugation; b=phosphoproteins isolated via ultracentrifugation; c=phosphopeptides isolated via EVTOP; and d=phosphoproteins isolated via EVTOP).

FIG. 7 is graphical data related to the enrichment efficiency of EVTOP beads by synthetic phosphopeptides (phos-iRT) (showing SEQ ID NOS: 1-5).

FIG. 8 illustrates the proteins-proteins interaction analysis of the up-regulated phosphoproteins in the PCNSL patient group, with each specified phosphoprotein and each black line indicating an upregulated phosphoprotein. The specified phosphoproteins are reported biomarkers for PCNSL or other diseases. The phosphoproteins represented by a black line are as follows with specified phosphoproteins bolded, starting with SPOCK2 and going clockwise: SPOCK2, HSPG2 (exterior GLCE and SULF2), GAL, SCG2, FSTL5, CHRDL1, FSTL3, APOA1, CFB, CIR, C4B, F5, C4A, POMC, IGFBP8, CD44, CAN, TFPI, AP0A2, AHSG, BMP6, CHRD, SPOCK3, STC2, IGFBP5, IGFBP6, KRT2, FLG, KRT1, KNGI, TRH, CHGB, PAM, 5CG5, PCSK9 (exterior EML4), HSP90B1 (exterior RCN1, NUCB1, and DNAJC3), APOE, RELN, RNXN3, CDH2 (exterior BOC), SPP1, IBSP, CFH, FBLN1, ECM1 (exterior ECM2), LAMB1, OLFM1, CLSTN1, C0L16A1, SP0N1, ADAMTS8, NCAN (exterior CNTN1), TNC, TNXB, PC0LCE, CST3, CTSB, CLU, LTBP2, LTBP1, VTN, C5, C18, C1QC (exterior ADAMDEC1 and FCGBP), CD14, STAB1, SCG3, CHGA, PRG4, F9, FGB, AP0A4, C3, C7, C8A, ESYT1, RPLP0, RPLP1, HABP4, NCL (exterior C3BP1), N0P56, C9, STRPING1, TTIB1, HRG, AP0B, LSR, APOL1, GSN, FGA, SEPP1, ALB (exterior KRT9 and ENPP2), RARRES2, CPB2, PAPLN, FBN1.

FIG. 9A is graphical data related to the intensities of HRG, GAK1B, LAMB1, and FIBB monitored in pre-chemotherapeutic samples and post-chemotherapeutic samples, with the zoomed view of phosphoprotein HRG illustrated in the inset.

FIGS. 9B and 9C show schematics of the proteins-proteins interaction and pathway analysis of phosphoproteins selected by machine learning (FIG. 9B) and same patients correlated intensity changes (FIG. 9C) of different phosphoproteins.

FIG. 10 is a table illustrating data related to an evaluation of EVTOP enrichment efficiency using synthetic phosphopeptides.

FIG. 11 is a table showing data related to sample results of phosphoprotein identification of CSF EVs isolated by cleaved EVTOP beads.

### LARGE TABLES

The following Large Tables are submitted herewith in ASCII plain text files in accordance with 37 C.F.R. § 1.52(c), the contents of which are incorporated by reference herein in their entireties:

Table S3 (entitled “TableS3.txt”; created Mar. 5, 2024; file size=53,206 bytes) is data relating to the results of phosphopeptide identification of cerebrospinal fluid (CSF) extracellular vesicles (EVs) isolated by cleaved EV to phosphoprotein (EVTOP) beads.

Table S4 (entitled “TableS4.txt”; created Mar. 5, 2024; file size=424,383 bytes) is data related to sample results of phosphopeptide identification from CSF EVs isolated by cleaved EVTOP beads.

Table S5 (entitled “TableS5.txt”; created Mar. 5, 2024; file size=27,853,057 bytes) is data related to results of the identified phosphoproteins of different volume of CSF using EVTOP method/beads.

Table S6 (entitled “TableS6.txt”; created Mar. 5, 2024; file size=1,409,388 bytes) is data related to results of the identified phosphopeptides of EVs isolated by EVTOP method/beads from different volumes of CSF.

Table S7 (entitled “TableS7.txt”; created Mar. 5, 2024; file size=338,015 bytes) is data related to results of the identified phosphoproteins of EVs isolated by ultracentrifugation method from different volumes of CSF.

Table S8 (entitled “TableS8.txt”; created Mar. 5, 2024; file size=3,017,228 bytes) shows the results of the identified phosphopeptides of EVs isolated by ultracentrifugation from different volumes of CSF.

Table S9 (entitled “TableS9.txt”; created Mar. 5, 2024; file size=309,548 bytes) is data relating to results of clinical CSF EV phosphoproteins isolated using the EVTOP method/beads.

Table S10 (entitled “TableS10.txt”; created Mar. 5, 2024; file size=3,331,566 bytes) is data relating to results of clinical CSF EV phosphopeptides isolated using the EVTOP method/beads.

Table S11 (entitled “TableS11.txt”; created Mar. 5, 2024; file size=45,632 bytes) is data relating to differential expression of clinical EV phosphoproteins.

Table S12 (entitled “TableS12.txt”; created Mar. 5, 2024; file size=88,663 bytes) are KEGG pathway and GO analysis results of upregulated phosphoproteins.

Table S13 (entitled “TableS13.txt”; created Mar. 5, 2024; file size=4,305 bytes) is data related to the phosphopeptides and phosphoproteins monitored via parallel reaction monitoring-parallel accumulation-serial fragmentation.

Table S14 (entitled “TableS14.txt”; created Mar. 5, 2024; file size=281,669 bytes) are quantitative results of monitored phosphopeptides of the 11 primary central nervous system lymphoma (PCNSL) patients in Example 7 below.

Table S15 (entitled “TableS15.txt”; created Mar. 5, 2024; file size=89,057 bytes) are quantitative results of phosphoproteins of the 11 PCNSL patients (the intensity of a phosphoprotein equals to the intensity summation of its corresponding phosphopeptides).

Each of the aforementioned tables can be found in Sun et al., Profiling phosphoproteome landscape in circulating extracellular vesicles from microliters of biofluids through functionally tunable paramagnetic separation, Angewandte Chemie 62(29): e202305668 (2023) and the Supporting Information thereto, all of which are incorporated by reference herein in their entireties.

Further, the entirety of the results sampled and referenced herein (including, without limitation, mass spectrometry phosphoproteomics raw and search files) have been deposited in a public repository, JPOSTrep, a member of ProteomXchange Consortium, accessible with the data set identifier PXD0036601 and JPST001838, the totality of which are incorporated herein by reference. Such data may also be submitted herewith and incorporated herein by reference in its entirety.

### DETAILED DESCRIPTION

For the purposes of promoting an understanding of the principles of the present disclosure, references will now be made to the embodiments illustrated in the drawings, and specific language will be used to describe the same. It will nevertheless be understood that no limitation of the scope of this disclosure is thereby intended.

A functionally tunable material is provided, as well as a method for leveraging the same, that allows for the recovery of phosphopeptides from cells and extracellular vesicles (EVs). The strategy employs a unique EV to phosphoproteins (EVTOP) platform, which is based on functionally tunable magnetic beads. The materials hereof allow for one-pot EV isolation, extraction and digestion of EV proteins, and enrichment of phosphopeptides using only a trace amount of biofluids.

Protein phosphorylation and de-phosphorylation play a fundamental role in both normal cell development and in a myriad of diseases and disorders. For example, the phosphorylation state of various proteins plays a central role in process such as apoptosis, aggressive growth, abnormal proliferation, the invasive growth of tumor cells, metastasis of cancer cells as well as normal cell growth, statis and death. Accordingly, there is a need to identify proteins that are phosphorylated in both normal and abnormal cells as well as tracking their phosphorylation states in various stages of cell growth, development and pathology.

Identification of disease biomarkers through mass spectrometry (MS)-based proteomics profiling, despite enormous promise, has been greatly limited due to the complexity of biofluids with an extremely wide dynamic range. EVs can offer an alternative solution to the challenge while having the attractive advantages of a liquid biopsy.

To date, a majority of research on EVs has concentrated on microRNAs, with only a small focus on EV proteins. The ability to detect functional proteins such as phosphoproteins can provide more useful real-time information about the organism's physiological status and disease progression, such as in the early detection and monitoring of cancers. The present inventors have previously reported in-depth analyses of phosphoproteomes in circulating EVs and demonstrated the feasibility of developing phosphoproteins as potential disease biomarkers. However, due to relatively low abundance of phosphoproteins and the requirement of additional sample processing steps for liquid chromatography (LC)-MS analyses, larger volume of biofluids have conventionally been required for initial discovery and development, which put severe constraint on the development of coherent, practical pipelines for systemic screening and validation of phosphoproteins using clinical biofluids.

Here, a novel strategy is provided that is based on a solid phase support scaffold that can be functionalized with various chemical groups. The solid phase support can comprise nanoparticles, magnetic beads, a polymer, silica beads, and/or the like. The scaffold tunable and can be used for multiple sample processing steps; from the isolation of Evs in biofluids (such as, for example, plasma, urine, saliva or CSF), EV protein extraction and digestion, to the enrichment of phosphopeptides for MS-based proteomic profiling, all in a single-tube, streamlined workflow. With the current format based on paramagnetic bead technology, EVTOP offers flexibility, scalability, and throughput. More importantly, EVTOP can simplify sample preparation, eliminate the need for offline cleanup steps, and minimize significant protocol optimization prior to proteome analysis. The data presented demonstrates the possibility of achieving ultrasensitive EV phosphoproteome analyses of challenging biofluids such as plasma and CSF with limited volume using the EVTOP approach hereof.

While the performance of EVTOP is shown in label-free quantitative data-dependent acquisition (DDA) measurements, it is conceivable that the strategy can be implemented for data-independent acquisition (DIA) measurements or for the commonly-used isotope labeling approaches.

As a specific subtype of non-Hodgkin lymphoma, PCNSL is confined to the central nervous system, which makes CSF the only ideal biofluid for accurate diagnosis. PCNSL is a highly heterogeneous disease and its pathogenesis is quite complex. Limited previous studies have shown that various factors can drive the occurrence of PCNSL and multiple signaling pathways may be aberrantly activated in patients with PCNSL, which can also lead to disease relapse. Based on EVTOP, a small volume of CSF samples from PCNSL patients can be analyzed and compared to healthy controls to identify disease biomarkers.

A “marker” or “biomarker” as the terms are used herein may be described as being differentially expressed when the level of expression in a subject who is experiencing an active disease state is significantly different from that of a subject or sample taken from a healthy subject or one not experiencing the disease state. A differentially expressed marker can be overexpressed or underexpressed as compared to the expression level of a normal or control sample, or subjects' baselines. The increase or decrease, or quantification of the markers in a biological sample, can be determined by any of the several methods known in the art for measuring the presence and/or relative abundance of a gene product or transcript. The level of markers can be determined as an absolute value, or relative to a baseline value, and the level of the subject's markers compared to a cutoff index. Alternatively, the relative abundance of the marker or markers can be determined relative to a control, which may be, for example, a clinically normal subject.

EVTOP and the related methods can be used for clinical screening and biomarker discovery dealing with large sample batches, limited sample material, and challenging biological specimens. While the advantages of EVTOP for EV phosphoproteome profiling with a trace amount of biofluids are shown, the strategy can be extended to the enrichment of EV peptides harboring other post-translational modifications provided the enrichment chemistries can be engineered to magnetic beads. Future implementations of EVTOP can include the enrichment of glycosylation, acetylation, and other modifications. Due to its ultra-sensitivity and robustness, EVTOP can be developed as a clinical procedure to screen or target functional proteins in circulating Evs for disease diagnosis.

In certain embodiments, EVTOP can be used to efficiently isolate EVs by magnetic beads functionalized with Ti(IV) ions and a membrane-penetrating peptide, octa-arginine R's, which can provide the hydrophilic surface to retain EV proteins during lysis. Subsequent on-bead digestion can concurrently convert EVTOP to Ti(IV) ion-only surface for efficient enrichment of phosphopeptides for phosphoproteomic analyses. As described herein, the streamlined, ultra-sensitive compositions and methods provided have allowed for the quantification of at least 500 unique EV phosphopeptides from only a few microliters of plasma and over 1,200 phosphopeptides with 100 μL of cerebrospinal fluid (CSF). The clinical applications of the compositions and methods hereof in evaluating the outcome of chemotherapy of primary central nervous system lymphoma (PCNSL) patients with small volume of CSF were also evaluated, which underscored that the compositions and methods hereof are powerful tools with broad clinical applications.

Leveraging these findings, a composition for recovery of phosphopeptides from cells and/or EVs is provided. The composition can comprise a solid phase support, at least a membrane binding/penetrating group (e.g., a cleavable membrane binding/penetrating group), and at least one metal ion or metal oxide. The at least one membrane binding/penetrating group and the at least one metal ion or metal oxide can be attached to the solid phase support. In certain embodiments, the cleavable membrane binding/penetrating group functionalizes a surface of the solid phase support and/or the at least one metal ion or metal oxide functionalizes the surface of the solid phase support.

The solid phase support can comprise a structure comprising a silica bead. The solid phase support can comprise a structure comprising a magnetic bead. The solid phase support can comprise a structure comprising a polymer bead or polymer resin. In certain embodiments, the solid phase support comprises a silica bead, magnetic bead, polymer bead, a polymer resin, or a combination of two or more of the foregoing. The solid phase support can comprise nanoparticles (e.g., magnetic nanoparticles, silica nanoparticles, and/or polymer nanoparticles (e.g., synthetic polymer nanoparticles)).

The solid phase support can, in certain embodiments, further comprise a silica and/or polymer shell coating at least a portion of the surface of the solid phase support. In certain embodiments, the solid phase support comprises polyethylene glycol (PEG). The PEG can comprise PEG_{20,000}, for example. In certain embodiments, the solid phase support can comprise PEG and a silica shell coating at least a portion of the surface thereof. This coating can then be functionalized with the at least one membrane binding/penetrating group and the at least one metal ion or metal oxide.

The metal ion or metal oxide can have an affinity for at least one phosphorylated residue in a phosphopeptide. The at least one metal ion or metal oxide can be positively charged.

In certain embodiments, the metal ion is selected from the group consisting of iron, copper, gallium, cobalt, nickel, calcium, zinc, cadmium, silver, palladium, platinum, and ruthenium. In certain embodiments, the metal ion is selected from the group consisting of titanium, zirconium, aluminum, vanadium, lead, manganese, and tin. The metal ion can be a Ti(IV) ion. In certain embodiments, the metal oxide is selected from the group consisting of titanium oxide, zirconium oxide and tin oxide, aluminum oxide, vanadium oxide, lead oxide and manganese oxide.

The at least one membrane binding/penetrating group can be a cleavable group and, in certain embodiments, has an affinity for binding a cell or an EV. The EV can be one or more of an exosome, a small EV (sEV), an ectosome, a microparticle, and/or a microvesicle.

A sEV can have a size of about 30 nm to about 100 nm (such as about 30 nm to 100 nm, 30 nm to 100 nm, or 30 nm to about 100 nm). For example, a sEV can have a size of about 35 nm to about 95 nm (such as about 35 nm to 95 nm, 35 nm to 95 nm, or 35 nm to about 95 nm). A sEV can have a size of about 35 nm to about 95 nm (such as about 35 nm to 95 nm, 35 nm to 95 nm, or 35 nm to about 95 nm). A sEV can have a size of about 40 nm to about 90 nm (such as about 40 nm to 90 nm, 40 nm to 90 nm, or 40 nm to about 90 nm). A sEV can have a size of about 45 nm to about 85 nm (such as about 45 nm to 85 nm, 45 nm to 85 nm, or 45 nm to about 85 nm). A SEV can have a size of about 50 nm to about 80 nm (such as about 50 nm to 80 nm, 50 nm to 80 nm, or 50 nm to about 80 nm). A sEV can have a size of about 55 nm to about 75 nm (such as about 55 nm to 75 nm, 55 nm to 75 nm, or 55 nm to about 75 nm). A sEV can have a size of about 60 nm to about 70 nm (such as about 60 nm to 70 nm, 60 nm to 70 nm, or 60 nm to about 70 nm). The ranges in this paragraph are inclusive of the stated end points and all 1 nm increments encompassed within the stated ranges. The membrane binding/penetrating group can be cleaved by, for example, an enzyme, a chemical, and/or by light. In certain embodiments, the membrane binding/penetrating group is selected from the group consisting of cell/membrane-penetrating peptides, membrane analogs, and membrane-bound molecules. The membrane binding/penetrating group can be a membrane-penetrating peptide. The membrane binding/penetrating group can be octa-arginine R⁺₈.

In certain embodiments, the at least one metal ion or metal oxide comprises a Ti(IV) ion and the binding/penetrating group comprises at least octa-arginine R⁺₈.

When subjected to an enzyme, a chemical, light, or a combination of any two or more of the foregoing, the binding/penetrating group can be cleaved from the solid phase support while the at least one metal ion or metal oxide remains attached to the surface of the solid phase support.

Kits for identifying phosphorylated peptides or the lack thereof (e.g., from EVs in a biofluid sample) are also provided. The EVs can comprise one or more of exosomes, sEVs, ectosomes, microparticles, and microvesicles. In certain embodiments, the kit comprises any variation of the composition described herein. In certain embodiments, the kit comprises a solid-phase support, at least a cleavable membrane binding/penetrating group, and at least one metal ion or metal oxide. The metal ion or metal oxide can have an affinity for at least one phosphorylated residue in a phosphopeptide. The at least one cleavable membrane binding/penetrating group can have an affinity for a cell or an EV. In certain embodiments, the at least one membrane binding/penetrating group and the at least one metal ion or metal oxide are attached to said solid phase support.

The solid phase support of the kit can comprise a structure comprising a silica bead. The solid phase support of the kit can comprise a structure comprising a magnetic bead. The solid phase support of the kit can comprise a structure comprising a polymer resin. In certain embodiments, the solid phase support of the kit comprises a silica bead, magnetic bead, and/or polymer resin. In certain embodiments, the solid phase support of the kit comprises a combination of two or more of a silica bead, a magnetic bead and a polymer resin. In certain embodiments, the silica bead and/or the magnetic bead are nanoparticles. In certain embodiments, the solid phase support of the composition of the kit comprises nanoparticles.

The metal ion of the composition of the kit can be selected from the group consisting of iron, copper, gallium, cobalt, nickel, calcium, zinc, cadmium, silver, palladium, platinum, and ruthenium. The metal ion of the composition of the kit can be selected from the group consisting of titanium, zirconium, aluminum, vanadium, lead, manganese and tin. The at least one metal ion or metal oxide of the composition of the kit can comprise a Ti(IV) ion. The at least one metal ion or metal oxide can be positively charged. The metal oxide of the composition of the kit can be selected from the group consisting of titanium oxide, zirconium oxide and tin oxide, aluminum oxide, vanadium oxide, lead oxide and manganese oxide.

The solid phase support of the composition of the kit can further comprise a silica and/or polymer shell coating at least a portion of the surface of the solid phase support. The solid phase support of the kit can further comprise polyethylene glycol and a silica shell coating at least a portion of the surface of the solid phase support.

The membrane binding/penetrating group of the kit can be cleaved by enzyme, a chemical, by light, or by a combination of any two or more of the foregoing. In certain embodiments, the binding/penetrating group is selected from the group consisting of cell/membrane-penetrating peptides, membrane analogs, and membrane-bound molecules. The binding/penetrating group can comprise at least octa-arginine R⁺₈, for example. In certain embodiments of the kit, the at least one metal ion or metal oxide of the composition comprises a Ti(IV) ion, and the binding/penetrating group comprises at least octa-arginine R's. In certain embodiments, the solid phase support of the kit is configured such that when is subjected to an enzyme, a chemical, light, or a combination of any two or more of the foregoing, the binding/penetrating group is cleaved from the solid phase support while the at least one metal ion or metal oxide remains attached to the surface of the solid phase support.

Methods for the one-pot capture and enrichment of peptides from a biofluid sample are also provided. Such methods can be employed to provide liquid biopsies and the like. In certain embodiments, such methods can be used for assessing therapeutic effect of a treatment in a subject.

In certain embodiments, the method uses the composition hereof to capture and enrich EVs (e.g., sequentially) in a biofluid sample collected from a subject. These steps can be performed in a one-pot reaction. As used herein, a “subject” can be a mammal, preferably a human, but can also be an animal.

In certain embodiments, the method comprises capturing EVs from a biofluid sample collected from a subject using any of the compositions described herein, lysing the captured EVs (e.g., by performing on-bead EV lysis through hydrophilic interaction liquid chromatography (HILIC), otherwise using light, using a chemical, or using another reagent), performing on-bead digestion to generate peptides, and cleaving the at least one membrane binding/penetrating group (e.g., R₈⁺) from the surface of the solid phase support (e.g., the EVTOP beads), thus leaving only the metal ions or metal oxide on the solid phase support which are bound to the peptide of interest (e.g., a phosphopeptide). In this manner, the method can achieve specific enrichment of specific peptides (e.g., phosphopeptides) for liquid chromatography with mass spectrometry (LC-MS) analyses.

Lysing the captured EVs can be performed through application of a reagent in an amount sufficient to lyse the captured EVs or by performing HILIC. In certain embodiments, the reagent comprises lithiumdodecyl sulfate.

The digesting step can be performed with Lys-C and trypsin, for example. Other enzymes known to those in the art can similarly be used for the digesting step. The length and temperature of the digestion can be varied to find the most optimal digestion condition.

The method can further comprise washing the non-phosphopeptides off of the solid phase support and, optionally, eluting the enriched phosphopeptides off of the solid phase support. Optimization of the EVTOP capability to capture phosphopeptides can also be achieved by varying the buffer conditions used to wash the non-phosphopeptides and/or to elute the phosphopeptides off of the EVTOP solid phase support (e.g., beads).

In certain embodiments, the method further comprises quantifying the generated peptides (e.g., phosphopeptides). The phosphopeptides can comprise, for example, one or more of the following: SPP1, Pro-thyrotropin-releasing hormone (TRH), Tenascin-C(TNC), Selenoprotein P (SEPP1), Fibrinogen beta chain (FGB/FIBB), Laminin subunit beta-1 (LAMB1), TNX, Histidine-rich glycoprotein (HRG), Golgi-associated kinase 1B (GAK1B) and Insulin-like growth factor-binding protein 5 (IGFBP5/IBP5).

In certain embodiments, the method further comprises identifying an upregulation or downregulation of the quantified phospholipids in the biofluid sample as compared to a control sample, and associating such upregulation or downregulation with the subject experiencing an active disease state or associating lack of upregulation or downregulation with the subject not experiencing the active disease state.

In certain embodiments, the method further comprises administering a treatment to the subject before or after collecting the biofluid sample from the subject. The terms “treat,” “treating,” “treated,” and “treatment” (with respect to a disease or condition, such as cancer) are used to describe a method for obtaining beneficial or desired results, such as clinical results, which can include, but are not limited to, one or more of improving a condition associated with a disease, curing a disease, lessening severity of a disease, increasing the quality of life of one suffering from a disease, prolonging survival and/or a prophylactic treatment. In reference to cancer, in particular, the terms “treat,” “treating,” “treated,” or “treatment” can additionally mean reducing the size of a tumor, completely or partially removing the tumor (e.g., a complete or partial response), stabilizing a disease, preventing progression of the cancer (e.g., progression-free survival), or any other effect on the cancer that would be considered by a physician to be a therapeutic or prophylactic treatment of the cancer. More particularly, curative treatment refers to any of the alleviation, amelioration and/or elimination, reduction and/or stabilization (e.g., failure to progress to more advanced stages) of a sign/symptom, as well as delay in progression of a sign/symptom of a particular disorder. Prophylactic treatment refers to any of the following: halting the onset, reducing the risk of development, reducing the incidence, delaying the onset, reducing the development, and increasing the time to onset of symptoms of a particular disorder. Desirable effects of treatment can include, but are not limited to, preventing occurrence or recurrence of a disease, alleviation of symptoms, diminishment of any direct or indirect pathological consequences of the disease, preventing metastasis, decreasing the rate of disease progression, amelioration or palliation of the disease state, and remission or improved prognosis. In some embodiments, compositions are used to delay development of a disease and/or tumor, or to slow (or even halt) the progression of a disease and/or tumor growth.

In certain embodiments the disease state is cancer. “Cancer” includes any neoplastic condition, whether malignant, pre-malignant or non-malignant. Generally, however, the neoplastic condition is malignant. Both solid and non-solid tumors are encompassed, and “cancer(ous) cell” may be used interchangeably with “tumor(ous) cell.” In certain embodiments, the cancer is PCNSL.

The treatment can comprise chemotherapy. The treatment can comprise radiation therapy. The treatment can comprise biopsy or surgery.

The method can comprise: (i) administering a treatment to the subject before collecting the biofluid sample from the subject; and (ii) quantifying the phosphopeptides to determine if the subject is experiencing an upregulation, downregulation, or normal expression of the phosphopeptides as compared to a control sample, wherein upregulation or downregulation is indicative of the subject experiencing an active disease state and normal expression is indicative of the subject not experiencing the active disease state. In certain embodiments, the phosphopeptides comprise one or more of the following: SPP1, Pro-thyrotropin-releasing hormone (TRH), Tenascin-C(TNC), Selenoprotein P (SEPP1), Fibrinogen beta chain (FGB/FIBB), Laminin subunit beta-1 (LAMB1), TNX, Histidine-rich glycoprotein (HRG), Golgi-associated kinase 1B (GAK1B) and Insulin-like growth factor-binding protein 5 (IGFBP5/IBP5).

Still further, the method can comprise collecting a first biofluid sample from the subject prior administering the treatment and a second biofluid sample from the subject after administering the treatment. Such biofluid samples can then be analyzed (e.g., using the compositions and EVTOP methods hereof). In such cases, downregulation of the phosphopeptides in the second biofluid sample as compared to the first fluid sample can be indicative of the treatment effectively treating the disease state. In this manner, the compositions and methods hereof can be used to assess treatment efficacy (or inefficacy), thereby enabling a healthcare provided to modify treatment if the desired effect is not achieved (or continue the present regimen if the present regimen is effective as indicated by the tests).

The biofluid sample (e.g., the first and/or second biofluid samples) can be plasma, urine, saliva, or CSF. In certain embodiments (of any of the methods hereof), a biofluid sample is CSF. In certain embodiments (of any of the methods hereof), a biofluid sample is urine. In certain embodiments (of any of the methods hereof), a biofluid sample is plasma. In certain embodiments (of any of the methods hereof), a biofluid sample is saliva.

The biofluid sample can comprise about 10 μL to about 200 μL of sample (such as about 10 μL to 200 μL, 10 μL to about 200 μL, or 10 μL to 200 μL). In certain embodiments, the biofluid sample comprises about 15 μL to about 195 μL of sample (such as about 15 μL to 195 μL, 15 μL to about 195 μL, or 15 μL to 195 μL). In certain embodiments, the biofluid sample comprises about 20 μL to about 180 μL of sample (such as about 20 μL to 180 μL, 20 μL to about 180 μL, or 20 μL to 180 μL). In certain embodiments, the biofluid sample comprises about 25 μL to about 175 μL of sample (such as about 25 μL to 175 μL, 25 μL to about 175 μL, or 25 μL to 175 μL). In certain embodiments, the biofluid sample comprises about 30 μL to about 170 μL of sample (such as about 30 μL to 170 μL, 30 μL to about 170 μL, or 30 μL to 170 μL). In certain embodiments, the biofluid sample comprises about 35 μL to about 165 μL of sample (such as about 35 μL to 165 μL, 35 μL to about 165 μL, or 35 μL to 165 μL). In certain embodiments, the biofluid sample comprises about 40 μL to about 170 μL of sample (such as about 40 μL to 170 μL, 40 μL to about 170 μL, or 40 μL to 170 μL). In certain embodiments, the biofluid sample comprises about 45 μL to about 165 μL of sample (such as about 45 μL to 165 μL, 45 μL to about 165 μL, or 45 μL to 165 μL). In certain embodiments, the biofluid sample comprises about 50 μL to about 160 μL of sample (such as about 50 μL to 160 μL, 50 μL to about 160 μL, or 50 μL to 160 μL). In certain embodiments, the biofluid sample comprises about 55 μL to about 155 μL of sample (such as about 55 μL to 155 μL, 55 μL to about 155 μL, or 55 μL to 155 L). In certain embodiments, the biofluid sample comprises about 60 μL to about 150 μL of sample (such as about 60 μL to 150 μL, 60 μL to about 150 μL, or 60 μL to 150 μL). In certain embodiments, the biofluid sample comprises about 65 μL to about 145 μL of sample (such as about 65 μL to 145 μL, 65 μL to about 145 μL, or 65 μL to 145 μL). In certain embodiments, the biofluid sample comprises about 70 μL to about 140 μL of sample (such as about 70 μL to 140 μL, 70 μL to about 140 μL, or 70 μL to 140 μL). In certain embodiments, the biofluid sample comprises about 75 μL to about 135 μL of sample (such as about 75 μL to 135 μL, 75 μL to about 135 μL, or 75 μL to 135 μL). In certain embodiments, the biofluid sample comprises about 80 μL to about 130 μL of sample (such as about 80 μL to 130 μL, 80 μL to about 130 μL, or 80 μL to 130 μL). In certain embodiments, the biofluid sample comprises about 85 μL to about 125 μL of sample (such as about 85 μL to 125 μL, 85 μL to about 125 μL, or 85 μL to 125 μL). In certain embodiments, the biofluid sample comprises about 90 μL to about 120 μL of sample (such as about 90 μL to 120 μL, 90 μL to about 120 μL, or 90 μL to 120 μL). In certain embodiments, the biofluid sample comprises about 95 μL to about 115 μL of sample (such as about 95 μL to 115 μL, 95 μL to about 115 μL, or 95 μL to 115 μL). In certain embodiments, the biofluid sample comprises about 100 μL to about 110 μL of sample (such as about 100 μL to 110 μL, 100 μL to about 110 μL, or 100 μL to 110 μL). All ranges described in this paragraph are inclusive of the stated end points and all 1 μL contained therein.

In certain embodiments, the biofluid sample comprises about 10 μL of plasma (such as 10 μL of plasma). In certain embodiments, the biofluid sample comprises about 100 μL of CSF (such as 100 μL of CSF). In certain embodiments, the biofluid sample comprises about 200 μL of urine (such as 200 μL of urine). In certain embodiments, the biofluid sample comprises about 50 μL of saliva (such as 50 μL of saliva).

Uses of the composition for assessing a physiological response in a subject (or lack thereof) to an administered treatment are also provided. The treatment can be chemotherapy. The treatment can be radiology. The treatment can be surgery (i.e., removal of a tumor). The treatment can be one or more of chemotherapy, radiology, and surgery.

All patents, patent application publications, journal articles, textbooks, and other publications mentioned in the specification are indicative of the level of skill of those in the art to which the disclosure pertains and incorporated herein by reference in their entireties.

In the above description, numerous specific details are set forth to provide a thorough understanding of the present disclosure. Particular examples may be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein may be applied to compounds and/or composition components that have configurations other than as specifically described herein. Indeed, it is expressly contemplated that the components of the composition and compounds of the present disclosure may be tailored in furtherance of the desired application thereof.

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein.

The term “about,” when referring to a number or a numerical value or range (including, for example, whole numbers, fractions, and percentages), means that the number or numerical range referred to is an approximation within experimental variability (or within statistical experimental error), and thus the numerical value or range can vary between 1% and 15% of the stated number or numerical range (e.g., +/−5% to 15% of the recited value), provided that one of ordinary skill in the art would consider equivalent to the recited value (e.g., having the same function or result).

When ranges are used herein for physical properties, such as molecular weight, or chemical properties, such as chemical formulae, all combinations and sub-combinations of ranges and specific embodiments therein are intended to be included.

The disclosure may be suitably practiced in the absence of any element(s) or limitation(s), which is/are not specifically disclosed herein. Thus, for example, each instance herein of any of the terms “comprising,” “consisting essentially of,” and “consisting of” (and related terms such as “comprise” or “comprises” or “having” or “including”) can be replaced with the other mentioned terms. Likewise, the singular forms “a,” “an,” and “the” include plural references unless the context clearly dictates otherwise. Thus, for example, references to “the method” include one or more methods and/or steps of the type, which are described and/or which will become apparent to those ordinarily skilled in the art upon reading the disclosure. The term “substantially” can allow for a degree of variability in a value or range, for example, within 90%, within 95%, or within 99% of a stated value or of a stated limit of a range.

It is recognized that various modifications are possible within the scope of the disclosure. Thus, although the present disclosure has been specifically disclosed in the context of preferred embodiments and optional features, those skilled in the art may resort to modifications and variations of the concepts disclosed herein. Such modifications and variations are considered to be within the scope of the disclosure as claimed herein.

It is therefore intended that this description and the appended claims will encompass all modifications and changes apparent to those of ordinary skill in the art based on this disclosure. For example, where a method of treatment or therapy comprises administering more than one treatment, compound, or composition to a subject, it will be understood that the order, timing, number, concentration, and volume of the administration is limited only by the medical requirements and limitations of the treatment (i.e., two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

Further, the use of headings and subheadings is for ease of reference, given the length of the document. Description under one heading or subheading (such as a subheading in the Detailed Description) is not intended to be limited to only the subject matter set forth under that particular heading or subheading.

### ENUMERATED EMBODIMENTS

Clause 1. A composition for recovery of phosphopeptides from cells and extracellular vesicles (EVs) comprising: a solid phase support; at least a cleavable membrane binding/penetrating group functionalizing a surface of the solid phase support, the cleavable membrane binding/penetrating group having an affinity for a cell or an EV; and at least one metal ion or metal oxide functionalizing the surface of the solid support, the metal ion or metal oxide having an affinity for at least one phosphorylated residue in a phosphopeptide.

Clause 2. The composition of clause 1, wherein the metal ion is selected from the group consisting of iron, copper, gallium, cobalt, nickel, calcium, zinc, cadmium, silver, palladium, platinum, and ruthenium.

Clause 3. The composition of clause 1, wherein the metal ion is selected from the group consisting of titanium, zirconium, aluminum, vanadium, lead, manganese and tin.

Clause 4. The composition of clause 1, wherein the at least one metal ion or metal oxide comprises a Ti(IV) ion.

Clause 5. The composition of clause 1, wherein the at least one metal ion or metal oxide is positively charged.

Clause 6. The composition of clause 1, wherein the metal oxide is selected from the group consisting of titanium oxide, zirconium oxide and tin oxide, aluminum oxide, vanadium oxide, lead oxide and manganese oxide.

Clause 7. The composition of clause 1, wherein the solid phase support comprises a silica bead, magnetic bead, a polymer resin, or a combination of any two or more of the foregoing.

Clause 8. The composition of any one of clauses 1-7, wherein the solid phase support comprises nanoparticles.

Clause 9. The composition of clause 1, wherein the solid phase support comprises nanoparticles.

Clause 10. The composition of any one of clauses 1-7 and 9, further comprising a silica and/or a polymer shell coating at least a portion of the surface of the solid phase support.

Clause 11. The composition of clause 1, wherein the solid phase support further comprises polyethylene glycol and a silica shell coating at least a portion of the surface of the solid phase support.

Clause 12. The composition of clause 1, wherein a membrane binding/penetrating group can be cleaved by an enzyme, a chemical, by light, or by a combination of any two or more of the foregoing.

Clause 13. The composition of any one of clauses 1-7 and 9, wherein the binding/penetrating group is selected from the group consisting of cell/membrane-penetrating peptides, membrane analogs, and membrane-bound molecules.

Clause 14. The composition of any one of clauses 1-7 and 9, wherein the binding/penetrating group comprises at least octa-arginine R⁺₈.

Clause 15. The composition of clause 1, wherein the at least one metal ion or metal oxide comprises a Ti(IV) ion, and the binding/penetrating group comprises at least octa-arginine R⁺₈.

Clause 16. The composition of clause 1, wherein when subjected to an enzyme, a chemical, light, or a combination of any two or more of the foregoing, the binding/penetrating group is cleaved from the solid phase support while the at least one metal ion or metal oxide remains attached to the surface of the solid phase support.

Clause 17. The composition of any one of clauses 1-7, 9 and 12, wherein the EVs comprise one or more of exosomes, small EVs (sEVs), ectosomes, microparticles, and microvesicles.

Clause 18. A kit for identifying phosphorylated peptides or the lack thereof, comprising: a solid phase support; at least a cleavable membrane binding/penetrating group functionalizing a surface of the solid phase support, the cleavable membrane binding/penetrating group having an affinity for a cell or an EV; and at least one metal ion or metal oxide functionalizing the surface of the solid support, the metal ion or metal oxide having an affinity for at least one phosphorylated residue in a phosphopeptide.

Clause 19. The kit of clause 18, wherein the solid phase support comprises a silica bead, magnetic bead, a polymer resin, or a combination of any two or more of the foregoing.

Clause 20. The kit of clause 18, wherein the metal ion is selected from the group consisting of iron, copper, gallium, cobalt, nickel, calcium, zinc, cadmium, silver, palladium, platinum, and ruthenium.

Clause 21. The kit of clause 18, wherein the metal ion is selected from the group consisting of titanium, zirconium, aluminum, vanadium, lead, manganese and tin.

Clause 22. The kit of clause 18, wherein the at least one metal ion or metal oxide comprises a Ti(IV) ion.

Clause 23. The kit of clause 18, wherein the at least one metal ion or metal oxide is positively charged.

Clause 24. The kit of clause 18, wherein the metal oxide is selected from the group consisting of titanium oxide, zirconium oxide and tin oxide, aluminum oxide, vanadium oxide, lead oxide and manganese oxide.

Clause 25. The kit of any one of clauses 18-24, wherein the solid phase support comprises nanoparticles.

Clause 26. The kit of clause 18, wherein the solid phase support comprises nanoparticles.

Clause 27. The kit of any one of claims 18-24 and 26, further comprising a silica and/or polymer shell coating at least a portion of the surface of the solid phase support.

Clause 28. The kit of clause 18, wherein the solid phase support further comprises polyethylene glycol and a silica shell coating at least a portion of the surface of the solid phase support.

Clause 29. The kit of clause 18, wherein a membrane binding/penetrating group can be cleaved by an enzyme, a chemical, by light, or by a combination of any two or more of the foregoing.

Clause 30. The kit of any one of clauses 18-24 and 26, wherein the binding/penetrating group is selected from the group consisting of cell/membrane-penetrating peptides, membrane analogs, and membrane-bound molecules.

Clause 31. The kit of any one of clauses 18-24 and 26, wherein the binding/penetrating group comprises at least octa-arginine R⁺₈.

Clause 32. The kit of clause 18, wherein the at least one metal ion or metal oxide comprises a Ti(IV) ion, and the binding/penetrating group comprises at least octa-arginine R⁺₈.

Clause 33. The kit of clause 18, wherein when subjected to an enzyme, a chemical, light, or a combination of any two or more of the foregoing, the binding/penetrating group is cleaved from the solid phase support while the at least one metal ion or metal oxide remains attached to the surface of the solid phase support.

Clause 34. The kit of any one of clauses 18-24, 26, 32, and 33, wherein the EVs comprise one or more of exosomes, sEVs, ectosomes, microparticles, and microvesicles.

Clause 35. A method of one-pot capturing and enriching phosphoproteins, phosphopeptides, or both from a biofluid sample, the method comprising: capturing EVs from a biofluid sample taken from a subject using a composition of any one of clauses 1-17; and enriching the captured EVs by: lysing the captured EVs while captured on the solid phase support, digesting the captured EVs to generate phosphopeptides, and cleaving the at least one membrane binding/penetrating group from the solid phase support. Clause 36. The method of clause 35, wherein lysing the captured EVs is performed through application of a reagent in an amount sufficient to lyse the captured EV.

Clause 37. The method of clause 36, wherein the reagent comprises lithiumdodecyl sulfate.

Clause 38. The method of any one of clauses 35-37, wherein digesting the captured EVs comprises digesting lysed proteins with Lys-C, trypsin, and/or other enzymes.

Clause 39. The method of clause 35, wherein digesting the captured EVs is performed for a time period and at a predefined temperature.

Clause 40. The method of clause 35, further comprising quantifying the phosphopeptides to determine if the subject is experiencing an upregulation, downregulation, or normal expression of the phosphopeptides as compared to a control sample, wherein upregulation or downregulation is indicative of the subject experiencing an active disease state and normal expression is indicative of the subject not experiencing the active disease state.

Clause 41. The method of clause 35, further comprising administering a treatment to the subject before or after collecting the biofluid sample from the subject.

Clause 42. The method of clause 41, wherein the treatment comprises chemotherapy.

Clause 43. The method of clause 41, further comprising quantifying the phosphopeptides to determine if the subject is experiencing an upregulation, downregulation, or normal expression of the phosphopeptides as compared to a control sample, wherein upregulation or downregulation is indicative of the subject experiencing an active disease state and normal expression is indicative of the subject not experiencing the active disease state.

Clause 44. The method of clause 40 or 42, wherein the disease state is cancer.

Clause 45. The method of clause 40 or 43, wherein the phosphopeptides comprise one or more of the following: SPP1, Pro-thyrotropin-releasing hormone (TRH), Tenascin-C(TNC), Selenoprotein P (SEPP1), Fibrinogen beta chain (FGB/FIBB), Laminin subunit beta-1 (LAMB1), TNX, Histidine-rich glycoprotein (HRG), Golgi-associated kinase 1B (GAK1B) and Insulin-like growth factor-binding protein 5 (IGFBP5/IBP5).

Clause 46. The method of clause 35, further comprising: administering a treatment to the subject before collecting the biofluid sample from the subject; quantifying the phosphopeptides to determine if the subject is experiencing an upregulation, downregulation, or normal expression of the phosphopeptides as compared to a control sample, wherein upregulation or downregulation is indicative of the subject experiencing an active disease state and normal expression is indicative of the subject not experiencing the active disease state; and wherein the phosphopeptides comprise one or more of the following: SPP1, Pro-thyrotropin-releasing hormone (TRH), Tenascin-C (TNC), Selenoprotein P (SEPP1), Fibrinogen beta chain (FGB/FIBB), Laminin subunit beta-1 (LAMB1), TNX, Histidine-rich glycoprotein (HRG), Golgi-associated kinase 1B (GAK1B) and Insulin-like growth factor-binding protein 5 (IGFBP5/IBP5).

Clause 47. The method of clause 46, further comprising collecting a first biofluid sample from the subject prior administering the treatment and a second biofluid sample from the subject after administering the treatment; wherein downregulation of the phosphopeptides in the second biofluid sample as compared to the first fluid sample is indicative of the treatment effectively treating the disease state.

Clause 48. The method of any one of clauses 35-47, wherein the biofluid sample is plasma, urine, saliva, or CSF.

Clause 49. The method of any one of clauses 35-48, wherein the biofluid sample is CSF.

Clause 50. The method of any one of clauses 35-49, wherein the biofluid sample comprises between about 10 μL to about 200 μL, inclusive of the end points.

Clause 51. The method of any one of clauses 35-48 and 50, wherein the biofluid sample comprises about 10 μL of plasma.

Clause 52. The method of any one of clauses 35-50, wherein the biofluid sample comprises about 100 μL of CSF.

Clause 53. The method of any one of clauses 35-48 and 50, wherein the biofluid sample comprises about 200 μL of urine.

Clause 54. The method of any one of clauses 35-48 and 50, wherein the biofluid sample comprises about 50 μL of saliva.

Clause 55. Use of the composition of any one of clauses 1-17 for assessing a physiological response in a subject, or lack thereof, to an administered treatment.

Clause 56. The use of clause 55, wherein the treatment is chemotherapy.

### EXAMPLES

The following examples serve to illustrate the present disclosure. The examples are not intended to limit the scope of the claimed invention in any way.

### Example 1: PpMB, TiMBM and EVTOP Synthesis

The strategy for extracellular vesicle (EV) to phosphoprotein (EVTOP) was devised with several unique features (Scheme 1): (i) EVTOP magnetic beads (MBs) were functionalized with Ti(IV) ions and a membrane-penetrating peptide, octa-arginine R⁺₈, to provide synergistic affinities for efficient EV isolation; (ii) positively charged Ti(IV) ions and R⁺₈ provide the hydrophilic surface for retaining EV proteins during on-bead EV lysis through hydrophobic-hydrophilic (HILIC) interaction (i.e., to retain the proteins on the solid phase support); (iii) on-bead digestion to generate peptides and concurrently cleave R₈⁺ off the beads; and (iv) with Ti(IV) ions only on the surface, the EVTOP beads hereof can achieve specific enrichment of phosphopeptides for liquid chromatography with mass spectrometry (LC-MS) analyses.

To prepare the various nanoparticles, one gram of PEG_{20,000}, 0.65 g of FeCl₃, and 1.2 g of sodium acetate were added successively into 20 mL of ethylene glycol with stirring (400 rpm/minute, magnetic stirring). Hunter, Signaling, Cell 100(1): 113-127 (2000). With the container sealed, the mixture was vigorously stirred at 60° C. for 1 hour and then transferred and reacted in a sealed Teflon-lined stainless-steel reaction kettle for 16 hours at 200° C. After the reaction, cooled to room temperature, the product (Fe₃O₄ nanoparticles) was washed with ethanol and H₂O by centrifugation at 5000 rpm and stored at 4° C. in H₂O. Next, 10 mg of Fe₃O₄ nanoparticles were dispersed in a mixed solution containing ethanol/H₂O/ammonia (160/40/3), and ultrasonically dispersed for 30 minutes. One milliliter of tetraethyl orthosilicate (TEOS) was added to the solution drop by drop, and the mixture was stirred (300 rpm/minute, mechanical stirring) for 6 hours at 30° C. to get Fe₃O₄@ SiO₂ nanoparticles. Sever et al., Signal Transduction in Cancer, Csh Perspect Medicine 5(4): a006098 (2015). The products were washed with ethanol and H₂O and solidificate at 90° C. for 1 hour. The obtained Fe₃O₄@SiO₂ nanoparticles were dispersed in H₂O and stored at 4° C. for further use. Then, 20 mg of Fe₃O₄@SiO₂ nanoparticles were dispersed in 200 mL of ethanol and 1 mL of aminopropyltriethoxysilane was added drop by drop, and the mixture was sonicated for 6 hours below 35° C. Subsequently, the amino-functionalized magnetic beads (Fe₃O₄@SiO₂—NH₂) were subjected to washing, solidification, and storing as aforementioned.

To prepare the Ti(IV) functional magnetic beads (Ti(IV) only), penetrating peptide (R₈, octa-arginines) functional magnetic beads (R's only), and EVTOP, the stock solutions were prepared first. The stock solution A comprising with 2.75 mg of 3-phosphonopropionic acid, 11 mg of N-Hydroxysuccinimide (NHS), 22 mg of 1-Ethyl-3-(3-dimethylaminopropyl) carbodiimide (EDC), and 5 mL of tris(hydroxymethyl)aminomethane (Tris)-HCl buffer at pH=6.5. Solution B consisted of 10 mg of R₈-PEG₂₀₀₀-COOH), 50 mg of NHS, 100 mg of EDC, and 5 mL of Tris-HCl buffer (pH=6.5). The carboxyl groups of R₈-PEG₂₀₀₀-COOH) and 3-phosphonopropionic acid were activated by EDC/NHS to react with the Fe₃O₄@SiO₂—NH₂.

To prepare Ti(IV) only beads, 1 mL of solution A was diluted with 4 mL of Tris-HCl buffer (pH=7.5), and 10 mg of Fe₃O₄@SiO₂—NH₂ nanoparticles was added. Shaken for 6 hours, the products were washed with H₂O three times and dispersed in 50 mL H₂O. Subsequently, 0.6 g of Ti(SO₄)₂ was added in the mixture and shaken for 1 hour.

For preparation of R's only beads, the pH of solution B was adjusted to 7.5 and 10 mg of Fe₃O₄@SiO₂—NH₂ nanoparticles was added to conduct the reaction for 6 hours.

To prepare EVTOP beads, 0.5 mL of solution A and B were mixed with 10 mg of Fe₃O₄@SiO₂—NH₂ nanoparticles (the pH was adjusted to 7.5), and the reaction occurred for 6 hours. Next, after three times washing with H₂O, 0.3 g of Ti(SO₄)₂ was poured in and mixed with shaking for 1 hour. Finally, the magnetic beads (MBs) were washed with H₂O three times and stored in 1 mL of 0.1% formic acid solution for further use.

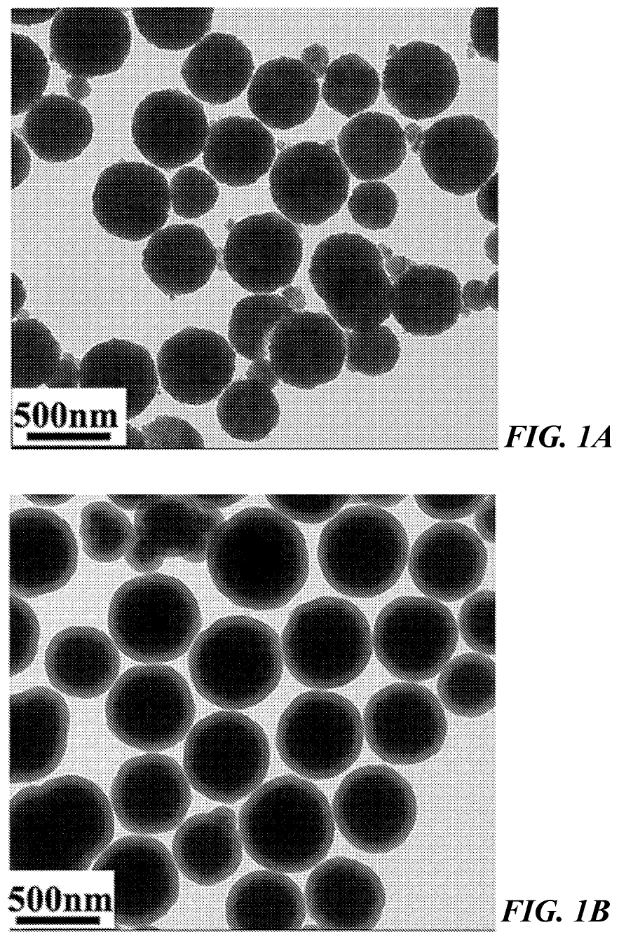

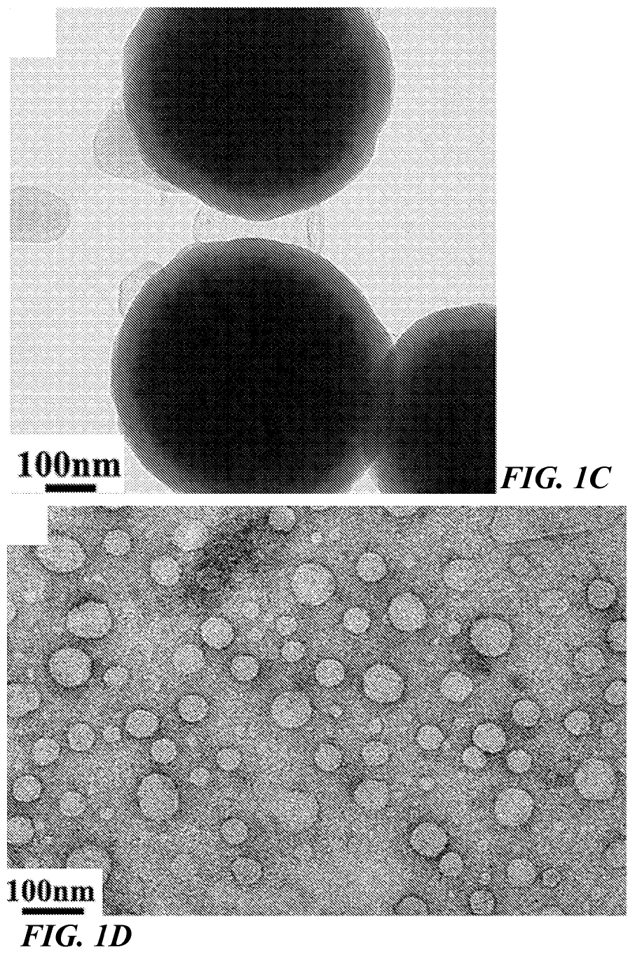

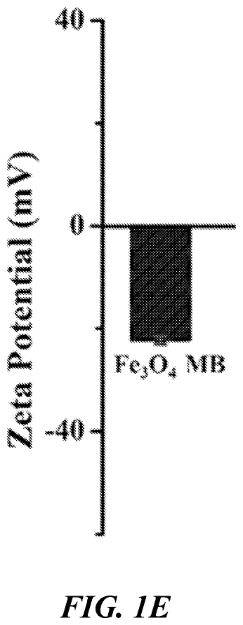

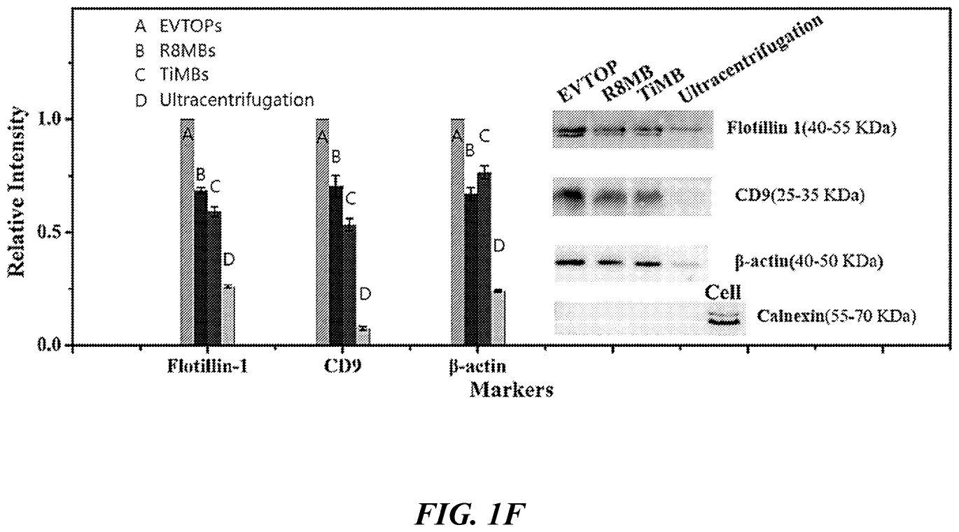

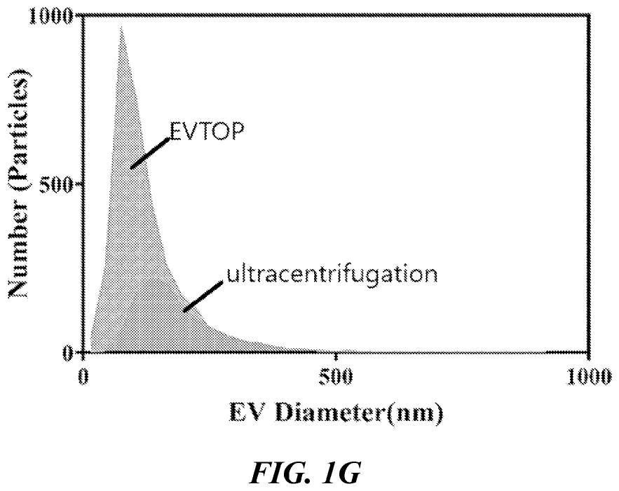

The morphology of the magnetic beads and magnetic beads coated with silica were characterized using the transmission electron microscopy (TEM) (Zeiss, Sigma; Zeiss Group, DE). Zetasizer™ (Malvern Panalytical, Malvern, UK) at each step of the modification process described herein, in part to investigate zeta potential. As shown in FIGS. 1A and 1B, a SiO₂ shell with a thickness of about 50 nm was clearly seen on surface of the prepared silica-shell-coated MBs (MBs@SiO₂).

Zeta potential was also characterized for each step during the preparation of Ti(IV) ion only, R's only, and EVTOP magnetic beads (FIG. 1E), with the successful introduction of each functionality leading to a zeta potential change. Due to the incorporation of PEG_{20,000} during the synthesis of the beads, the ether bond from polyethylene glycol (PEG) carried a weak negative charge in solution, which reduced the potential value of beads as compared to beads without incorporation of PEG_{20,000}, and subsequently influenced the beads during silica coating and molecule modification.

### Example 2: EV Isolation from Biofluids

A key feature of EVTOP is the ability to achieve all sample preparation steps starting from a biofluid to enriched EV phosphopeptides through interactions and conversion of functional groups on the same MBs. The one-pot approach without any cleanup or desalting steps prior to mass spectrometer (MS) analysis can minimize sample loss and allow for extremely sensitive and quantitative phosphoproteomics with microliters of clinical samples.

Urinary, plasma, salivary and cerebrospinal fluid (CSF) samples were obtained with the approval of Huashan Hospital Affiliated to Fudan University, Shanghai, China. These samples were processed and used according to the ethical guidelines of the hospital, and the appropriate agreement of all patients were obtained prior to sample collection. CSF samples from healthy controls and primary central nervous system lymphoma (PCNSL) patients were obtained and centrifuged at 2,500 g for 15 minutes at room temperature to remove cell debris, large aggregates, and apoptotic bodies, and repeated the operation once. The processed biofluids were collected and frozen at −80° C. until used.

The CSF, saliva, urine and plasma samples were thawed at physiological conditions, diluted using equal amount of phosphate-buffered saline (PBS) and centrifuged with 12,000 g at 4° C. for one hour. Subsequently, the supernatant was further subjected to ultracentrifuge (Beckman) to get the pellet after 120,000 g centrifugation. The cooled PBS was then added to disperse the pellets after another 1 hour of ultracentrifugation process. The aforementioned procedure was repeated and collected pellets were stored at −80° C.

The relative efficiency of EV isolation from CSF and other biofluids was then investigated, with western blotting (WB) experiments carried out to examine EV capture by three functionalized beads (wherein EVTOP beads are employed, termed the “EVTOP method”), along with the ultracentrifugation method for comparison purposes.

For EV isolation, two times volume of 0.01% NP40/TritonX-100 in PBS was added to CSF (four times of 0.01% NP40/TritonX-100 in PBS for saliva, ten times of 0.01% NP40/TritonX-100 in PBS for plasma, and one-tenth volume of 0.1% NP40/TritonX-100 in PBS for urine) prior to the addition of EVTOP beads (or the other two types of MBs). While CSF was used in this study, it will be understood that the data can translate to other biofluids. After 1 hour of incubation at room temperature, the beads were washed three times with PBS, separated with a magnet, and the supernatant was removed. In a separate group, the EVs were kept immobilized on the surface of the EVTOP beads, R⁺₈ only MBs, and Ti(IV) only MBs without elution.

The images of EVs immobilized on the surface of EVTOP beads and the eluted EVs were obtained by TEM (HITACHI H-8100, Hitachi, Japan) under a 200 KV potential to assess the number or volume distribution of nanoparticles as a function of their size. The EV samples were incubated in 2% (w/v) phosphotungstic acid solution for 10 minutes, put on 200-mesh formvar carbon-coated copper grids and dried at ambient conditions.

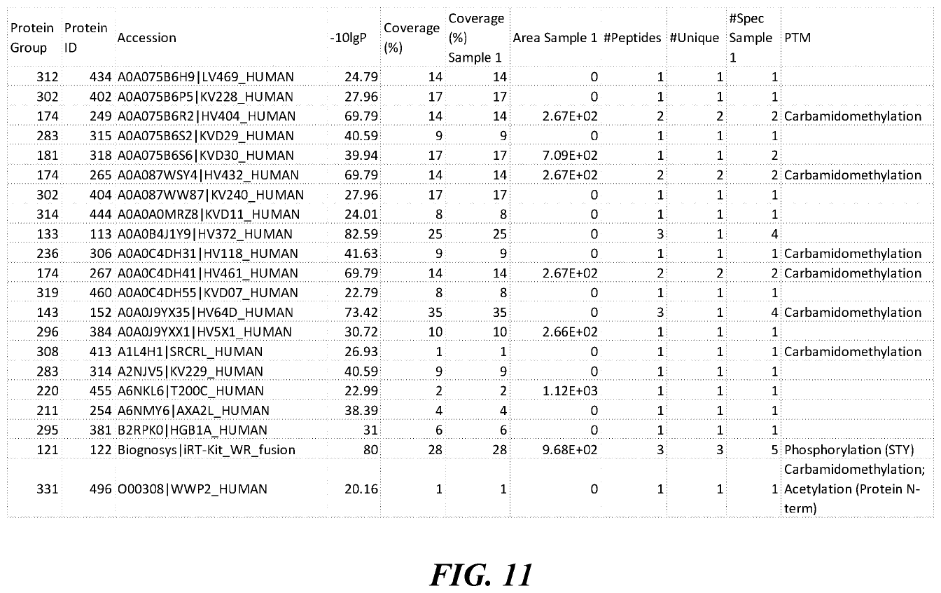

TEM images of EVTOP beads capturing EVs isolated from CSF and the eluted CSF EVs are shown in FIGS. 1C and 1D, respectively. The transparent membrane structure of EVs adhered to the surface of EVTOPs was clearly observed and the morphology of EVs remained intact after elution. Data related to the phosphopeptides identified from the CSF EVs isolated by cleaved EVTOP beads are shown in Table S4, while data related to phosphoproteins identified from CSF EVs isolated by cleaved EVTOP beads are shown in FIG. 11 and Table S3.

For WB analysis, each EV sample was isolated from 0.5 mL of CSF by different types of beads (Ti(IV) only MBs, R⁺₈ only MBs, and EVTOP beads) or via ultracentrifugation and, thereafter, assessed subjected to liquid chromatography with tandem mass spectrometry (LC-MS/MS) analysis.

The EVs separated by the different methods were then lysed using 20 μL of lithiumdodecyl sulfate (LDS) loading buffer (1×) at 95° C. for 5 minutes. Subsequently, the lysed EVs were loaded into the lanes of 15% sodium dodecyl sulfate polyacrylamide gel electrophoresis (SDS-PAGE) (EpiZyme, Shanghai, China) employing 190 V for 70 minutes. Following SDS-PAGE, the lysed EVs were transferred onto polyvinylidene fluoride (PVDF) membranes (Millipore, Sigma, Sigma-Aldrich, St. Louis, MO) by a current at 275 mA for 70 minutes, and the resulting PVDF membranes were blocked using TBS containing 0.2% TritonX-100 (w/v) (TBST) and 1% bis(trimethylsilyl) acetamide (BSA) (w/v) for 1 hour.

Further, rabbit anti-CD9 (D3H4P, Cell Signaling Technology) at 1:2500 v/v, mouse anti-β actin (ab8226, Abcam) at 1:2500 v/v, and rabbit anti-Flotillin 1 (ab133497; Abcam, Inc., Boston, MA) at 1:2500 v/v were added into TBST containing 1% BSA to incubate with the PVDF membranes overnight in a refrigerator at 4° C. After washing three times each, 1 μL of HRP functional secondary antibody was added to combine with the antibody on the surface of the PVDF membranes specifically in the TBST with 1% BSA for 1 hour. Finally, quantitative assay was conducted by exposure with an enhanced chemiluminescence imager (ImageQuant LAS500, GE HealthCare Life Sciences, Inc., Marlborough, MA).

Based on the WB results using three EV markers (CD9, flotillin 1, and β-actin), the order of EV isolation efficiency was determined as EVTOP>R⁺₈ only >Ti(IV) only >ultracentrifugation, with the EVTOP methodology being most efficient (FIG. 1F).

For LC-MS/MS processing, EVs isolated from different biofluids underwent lysis, denaturing, reduction, and alkylation by introducing 10 μL of lysis buffer comprising 1 μL of 120 mM sodium deoxycholate, 1 μL of 120 mM sodium lauroyl sarcosinate, 1 μL of 120 mM 2-Chloroacetamide, 0.2 μL of Tris (2-carboxyethyl) phosphine, 0.2 μL of Tris (2-carboxyethyl) phosphine, 0.1 μL of phosphatase inhibitor cocktail, 5.7 μL of H₂O, and were then heated at 95° C. for 10 minutes. After cooling to room temperature, 40 μL of 50 mM triethylammonium bicarbonate was added followed by the digestion using Lys-C (Wako, enzyme to protein ratio of 1:100) at 37° C. for 3 hours. Subsequently, trypsin was added in enzyme-to-protein of ratio of 1:50 (w/w) and the same conditions were maintained for 6 hours. Thereafter, 5.5 μL of 10% (v/v) trifluoroacetic acid was introduced to terminate the digestion, followed by the addition of ethyl acetate in equal amounts.

The mixture was vortexed for 3 minutes and centrifuged at 15,000 g to separate the aqueous and organic layers. The upper layer was discarded and the aqueous layer was utilized for phosphopeptide enrichment.

In case of EVTOP beads group, after the top later was removed, 60 μL of 200 mM GA in ACN/TFA (98:2, v/v) were added to the solution, followed by shaking at 1500 rpm (26° C.) for 20 minutes. EVTOP beads along with enriched phosphopeptides were separated by a magnet and washed tandemly with 100 mM GA in ACN/H₂O/TFA (50:49:1, v/v/v), 25 mM GA in ACN/H₂O/TFA (80:19.8:0.2, v/v/v), and 80% ACN in H₂O.

Finally, the enriched phosphopeptides were eluted with 50 μL of 400 mM NH₄OH twice and dried with a CentriVap freeze concentrator (Labconco Corporation, Kansas City, MO).

For the samples treated with ultracentrifugation, the obtained EVs were subjected to lysis, digestion, drying and then desalting using 2 mm extraction disks (3M Empore 2240-SDB-XC (Thermo Fisher, Waltham, MA)) following the manufacturer's protocol and freeze dried again. After re-dissolution, PolyMAC Phosphopeptides Enrichment Kit (Tymora Analytical Operations, West Lafayette, IN) was employed for the enrichment of phosphopeptides. The freeze-dried product was then redissolved in 0.1% formic acid and carried to LC-MS/MS analysis.

Target LC-MS/MS analysis was performed by a trap-elute mode on a NanoLC 400 Systems system (Eksigent Technologies LLC, Dublin, CA) coupled to a 6500 QTRAP mass spectrometer (AB Sciex, Foster City, CA) by a nanospray ion source (AB Sciex, Foster City, Singapore). Mobile phases consisted of 0.1% formic acid (FA) in 2% ACN solution (Solution A) and 98% ACN solution with 0.1% FA (Solution B). 1 μL of sample was loaded on a 200 mm*0.5 mm ChromXP C18-CL 3 μm 120 Å column (Eksigent Technologies LLC, Dublin, CA) by holding at 100% Solution A for 7 minutes with 3 μL/minutes, with the column temperature at room temperature (25° C.). The sample was then subjected to the analytical column and separated by a home-made column (inner diameter as 150 μm, length as 20 cm, C18 resin as 1.9 μm) using the following gradient method: gradient from 5 to 20% Solution B for 5 minutes, gradient from 20 to 30% Solution B for 13 minutes, gradient from 30 to 70% Solution B for 7 minutes, holding 70% Solution B for 20 minutes, gradient from 70 to 5% Solution B for 0.1 min, re-equilibrating at 5% Solution B for 5 minutes. The flow rate was 300 nL/minute and the column temperature was at room temperature.

The MS was used in positive ion mode with parameters consisting of a 2000 V Nanospray voltage, curtain gas setting of 20, nebulizer gas setting of 0, and an interface heater temperature of 100° C. Collision energy (CE) was set at the optimal value (see Table 1 below). Entrance potential (EP) was set to 10, cell exit potential (CXP) was set to 13, and Q1 and Q3 were set to unit/unit resolution (0.7 Da).

The size distribution and concentration of EVs isolated by EVTOP method was also evaluated and the ultracentrifugation method using nanoparticle tracking analysis (NTA) and TEM characterization. The EVs used for NTA and TEM characterization were eluted from beads via shaking after the addition of 100 μL (100 mM) of triethylamine. The operation was repeated once, a magnet was used to separate the magnetic beads (where applicable), and the collected supernatant was transferred into a new centrifuge tube.

More specifically, for NTA, the isolated EVs from 1 mL of CSF specimen were eluted using 0.1 mL of 0.1 M triethylamine and diluted to 1 mL adding PBS. The size of EVs was examined using the calibrated ZetaView (Particle Metrix, Meerbusch, Germany). The instrumental parameters were set as brightness to 20, sensitivity to 70, and shutter to 100. The reported values are the average of three replicates.

The EVTOP method isolated 1.61×e⁹ particles/mL with the average size of 125.6 nm and 2.57×e⁸ particles/mL with the average size of 161.4 nm for the ultracentrifugation method, from the equal volume of CSF (FIG. 1G).

### Example 3: Tunable Bifunctional Group Evaluation & Capture Efficiency Studies

To rationalize the design of EVTOP beads with tunable bifunctional groups (e.g., metal ions and at least one cleavable membrane binding/penetrating group), the EVTOP beads were compared with the other MBs singly functionalized with Ti(IV) only or with R⁺₈ only—for example in comparative evaluation with respect to both EV isolation and phosphopeptide enrichment.

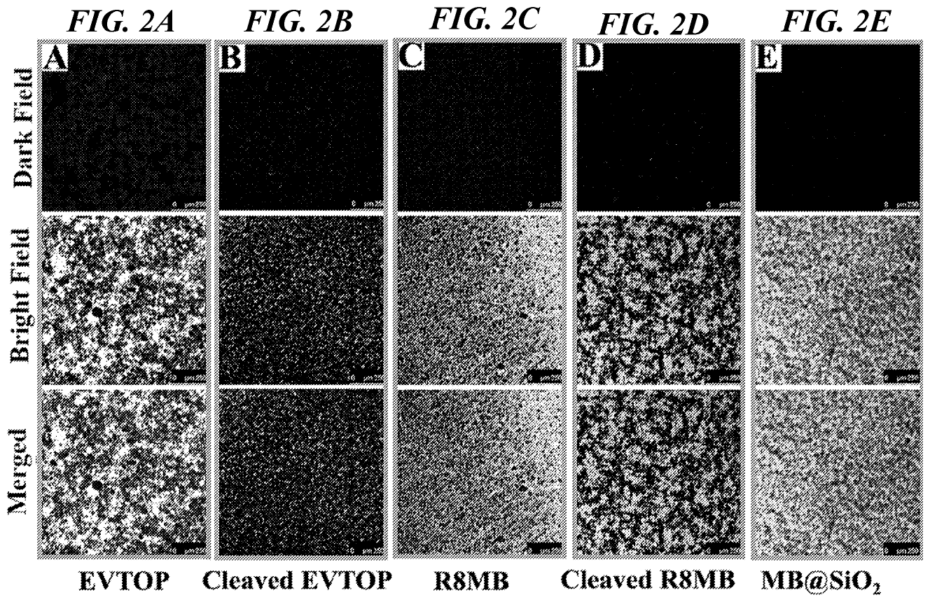

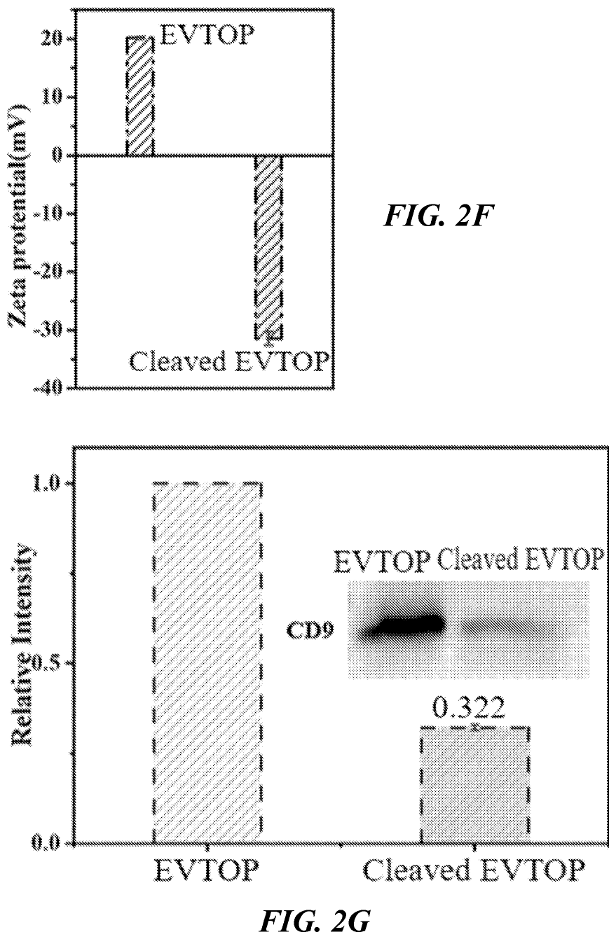

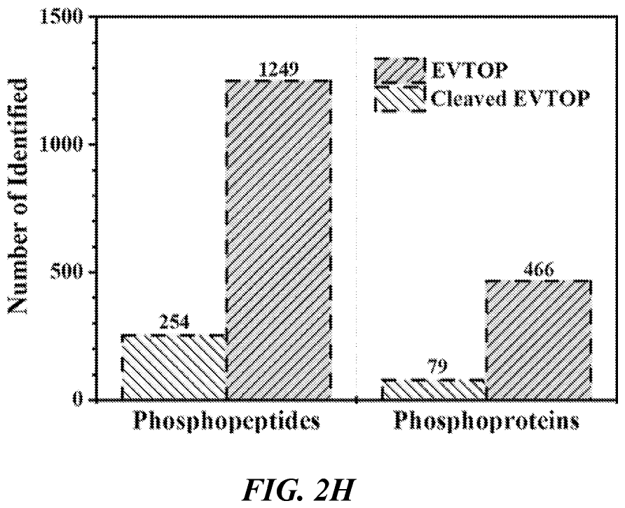

First, EVTOP beads and MBs functionalized with R⁺₈ only (prepared as described above in Example 1) were first treated with trypsin overnight at 37° C., followed by the incubation with CSF to capture EVs (FIG. 2A (EVTOP beads) and FIG. 2C (R⁺₈ MBs only)). 1,1′-Dioctadecyl-3,3,3′,3′-tetramethyl indodicarbo-cyanine-5,5′-disulfonic acid (DiD) fluorophore was also added to directly visualize the EVs captured on the functionalized beads.

More specifically, the EVs immobilized on each set of beads were dispersed in PBS, incubated with the cell membrane dye DiD at the ratio of 1000:1 (v/v) for 20 minutes at room temperature in dark and then washed with PBS three times. For consistency, 0.5 mL of CSF and an equal amount of beads from each category were used per sample. The beads were concentrated by magnet before each fluorescence image was taken by LSM700 laser scanning microscope (Zeiss, Heidenheim, Germany).

As shown in FIGS. 2A and 2B, EVTOP beads after trypsin digestion showed obvious decrease in the capture efficiency, while the EV capture efficiency of trypsin-treated R⁺₈ only MBs and silica coated only MBs dropped to almost zero (FIGS. 2C-2E). A zeta potential change due to the trypsin treatment was also detected, as expected (FIG. 2F).

For the EVTOP beads, EVs isolated from different biofluids underwent lysis, denaturing, reduction, and alkylation as described above in Example 2 for LC-MS/MS processing.

For ultracentrifugation, the EVs obtained were subjected to lysis, digestion, drying and then desalting as described above in Example 2.

WB and MS identification of phosphopeptides (performed as described above) further confirmed the important role of R's and Ti(IV) ions. By concurrently cleaving R⁺₈ off during trypsin digestion, EVTOP beads were converted to Ti(IV) ion-only beads, which led to more efficient and selective phosphopeptide enrichment (FIGS. 2G and 2H).

### Example 4: Protein Phosphorylation Profiling from Trace Amounts of Biofluids

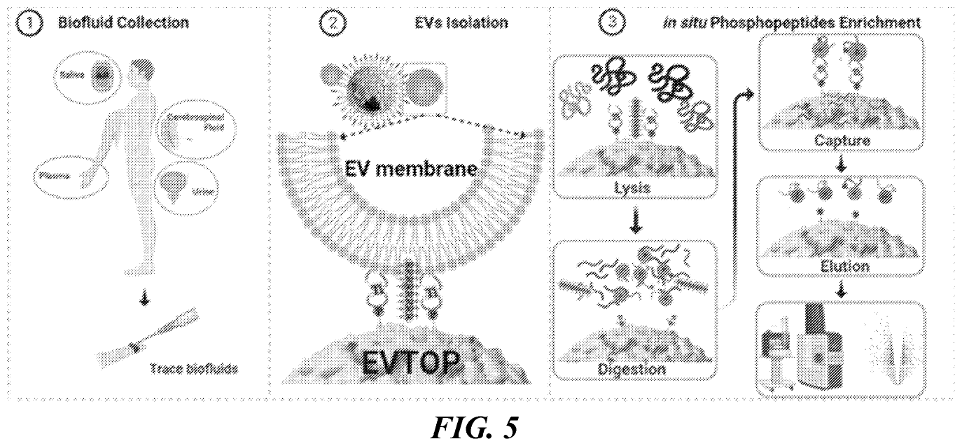

FIG. 5 shows a scheme for isolation of EVs from trace amounts of biofluids and in situ attachment of the phosphopeptides for in-depth phosphoproteome profiling by EVTOP beads.

It is well known that sampling CSF, typically through a spinal tap, is difficult and the volume is much smaller than that of other biofluids such as saliva, urine, and plasma. About 80% of the proteins in CSF come from the plasma ultrafiltrate that passes through the choroid plexus blood-brain barrier (BBB) in the lateral ventricle, with the remaining 20% coming from the drainage of the central nervous system (CNS) interstitial fluid. Furthermore, the concentration of the proteins in CSF is only about 1% of that in plasma, making it harder to get enough information on the phosphoproteome of CSF or CSF EVs. Ueno et al., Blood-brain barrier and blood-cerebrospinal fluid barrier in normal and pathological conditions, Brain Tumor Pathologies 33 (2): 89-96 (2016); Ogata et al., Evaluation of protein depletion methods for the analysis of total-, phospho- and glycoproteins in lumbar cerebrospinal fluid, J Proteomic Research 4 (3): 837-845 (2005). Previous studies based on CSF phosphoproteomics have had limited success.

Clinical samples are typically limited by their quantities. As such, the present EVTOP beads were tested using trace amounts of biofluid samples to demonstrate clinical application. More specifically, the one-pot EVTOP method (e.g., EVTOP beads) described above was used to streamline the sample preparation, followed by EV phosphoproteomic analyses performed as described herein with plasma, CSF, saliva, and urine samples. To compare the EVTOP method with conventional procedures, the analysis was repeated using ultracentrifugation followed by standard lysis, protein extraction, digestion and phosphopeptide enrichment.

Further, in a separate analysis, different volumes (from 100 μL to 500 μL) of CSF were used to investigate the phosphopeptides enrichment efficiency.

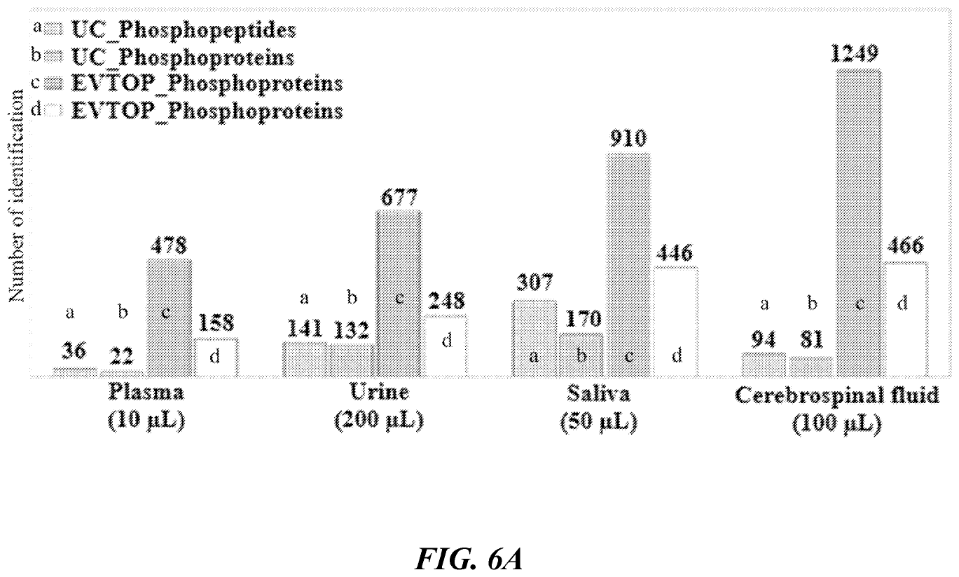

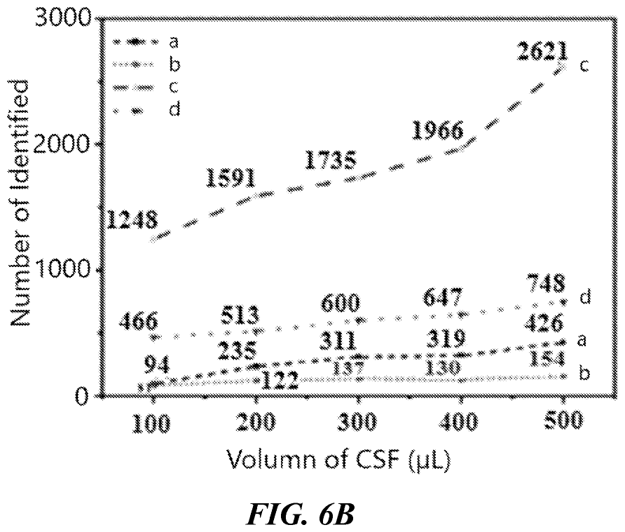

As shown in FIG. 6A, with 10 μL of plasma, 478 phosphopeptides corresponding to 158 phosphoproteins were identified, 677 phosphopeptides corresponding to 248 phosphoproteins from 200 μL of urine, 910 phosphopeptides corresponding to 446 phosphoproteins from 50 μL of saliva, and 1,249 phosphopeptides corresponding to 466 phosphoproteins from 100 μL of CSF. In contrast, the conventional procedure resulted in extremely poor results with the same amounts of starting biofluids (FIG. 6A). For example, only 94 phosphopeptides were identified from 100 μL of CSF and 36 phosphopeptides with 10 μL of plasma.

Analysis using different volumes (from 100 μL to 500 μL) of CSF to assess phosphopeptide enrichment efficiency further supported the superior performance of EVTOP beads as compared to conventional techniques (FIG. 6B) (see Tables S5 and S6 related to phosphoproteins and phosphopeptides, respectively, isolated by EVTOP beads from different volumes of CSF; see Tables S7 and S8 related to phosphoproteins and phosphopeptides, respectively, isolated by ultracentrifugation from different volumes of CSF).

### Example 5: Enrichment Efficacy Studies

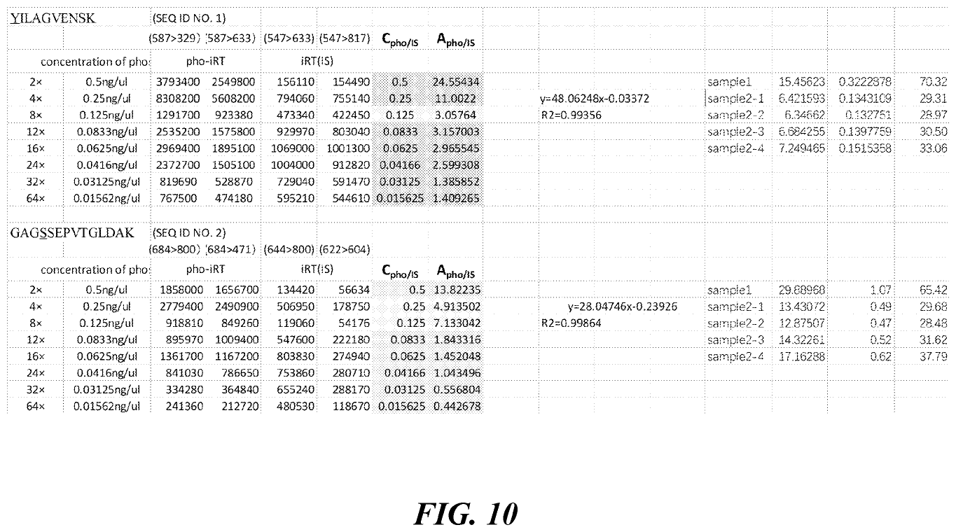

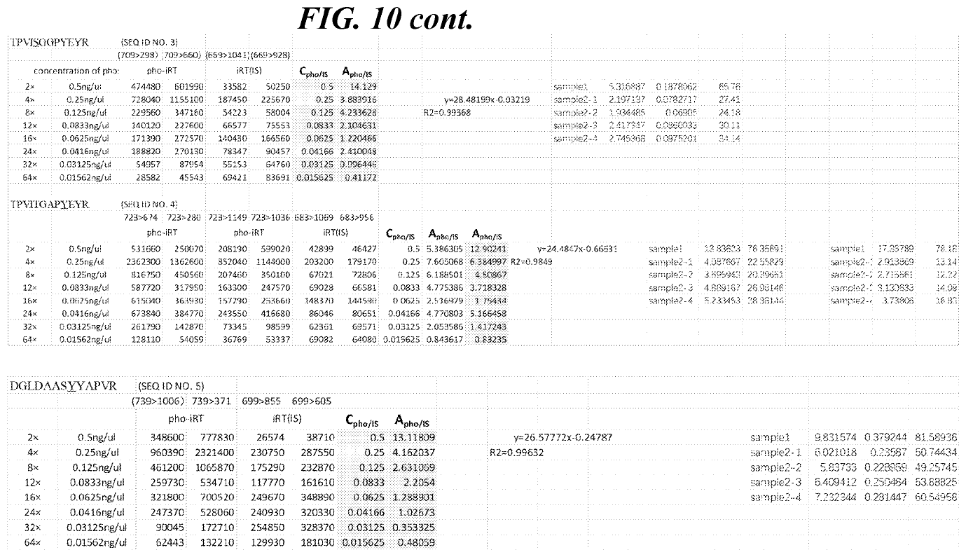

To quantify the enrichment efficacy of EVTOP beads, a multiple reaction monitoring (MRM-MS) method was used a on triple quadrupole mass spectrometer using multiple synthetic phosphopeptides (Table 1 and FIG. 10). The five synthetic phosphopeptides were designed and synthesized with non-naturally occurring peptide sequences. Synthetic phosphopeptides were added directly into CSF samples or into the lysate after EVs were captured by EVTOP beads from CSF and lysed on the beads as described above. Thereafter, the synthetic phosphopeptides were measured in each sample using MRM-MS.

TABLE 1
MRM parameters of target phosphopeptides

| Peptide | Precursor | Qualitative | Quantitative | | Dwell |
|---|---|---|---|---|---|
| sequence | ion (m/z) | ion (m/z) | ion (m/z) | CE(V) | time (ms) |
| YILAGVENSK | 587 | 633 | 329 | 36.8 | 20 |
| (SEQ ID NO: 1) | | | | | |
| GAGSSEPVTGLDAK | 684 | 471 | 800 | 32.6 | 20 |
| (SEQ ID NO: 2) | | | | | |
| TPVISGGPYEYR | 709 | 660 | 298 | 33.8 | 20 |
| (SEQ ID NO: 3) | | | | | |
| TPVITGAPYEYR | 723 | 1149 | 674 | 37.2 | 20 |
| (SEQ ID NO: 4) | | | | | |
| DGLDAASYYAPVR | 739 | 371 | 1006 | 29.2 | 20 |
| (SEQ ID NO: 5) | | | | | |

More specifically, the five synthetic phosphopeptides according to the sequences of iRT (phos-iRT) were set as target analytes, and the corresponding peptides of iRT with the same sequence were used as internal standards. The signals of iRT and phos-iRT were acquired by using 2 transitions (qualitative and quantitative transitions) of each peptide. Peak areas of transitions that corresponded with the iRT and phos-iRT were quantified using Skyline software by subtracting the area of the blank sample. A standard curve was established with 6-point calibration curves (n=3) that ranged from 0.01 to 0.5 ng/μL for phos-iRT (0.1% FA added) and dissolved in 9 μL 2000×iRT. The peak area ratio of phosphorylated peptide to the corresponding peptide with the same sequence was used as Abscissa, and the concentration ratio of phosphopeptide to corresponding iRT peptide was used as ordinate.

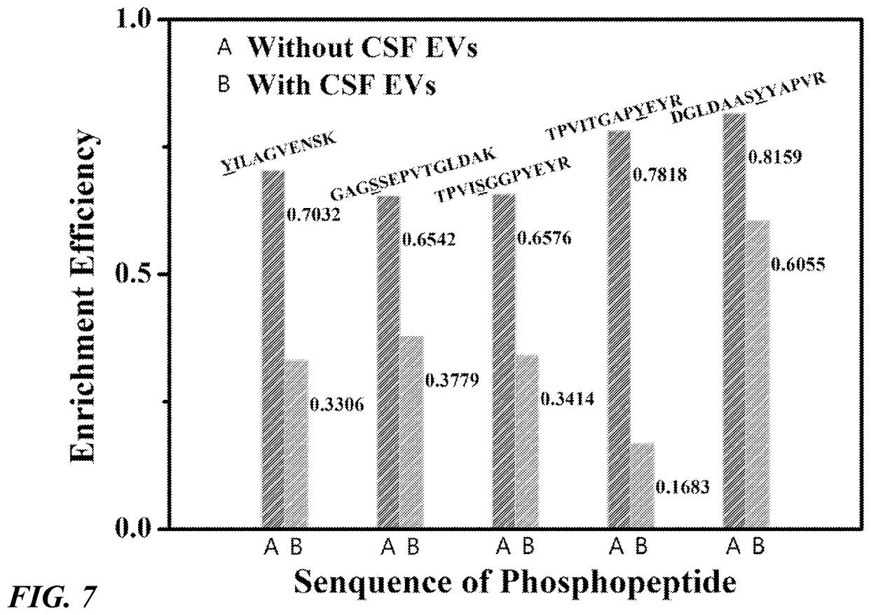

An average of 84% of the synthetic phosphopeptides added in the lysis step were recovered (i.e., about 84% sample recovery yield from lysate to phosphopeptides), and approximately 40% of the synthetic phosphopeptides were recovered from the CSF EV sample (i.e., about 40% yield from EV samples to phosphopeptides) (FIG. 7).

### Example 6: EVTOP Clinical Applications

The ability to use a small amount of CSF samples to achieve sensitive EV phosphoproteomics is particularly appealing as the development of CSF-based diagnosis has been limited by its available quantity for many diseases. For example, PCNSL is a rare subtype of non-Hodgkin lymphoma and is confined to the CNS. As a highly heterogenous disease with a complex pathogenesis, the molecular characterization of PCNSL could be critical. In addition, there are many benefits to using CSF as opposed to other biofluids as, for example, CSF is often a less-invasive biofluid option for accurate disease evaluation. However, previous attempts using conventional techniques on a large volume of CSF samples only identified a small number of phosphoproteins. Bahl et al., Characterization of the human cerebrospinal fluid phosphoproteome by titanium dioxide affinity chromatography and mass spectrometry, Analytical Chemistry 80(16): 6308-6316 (2008).

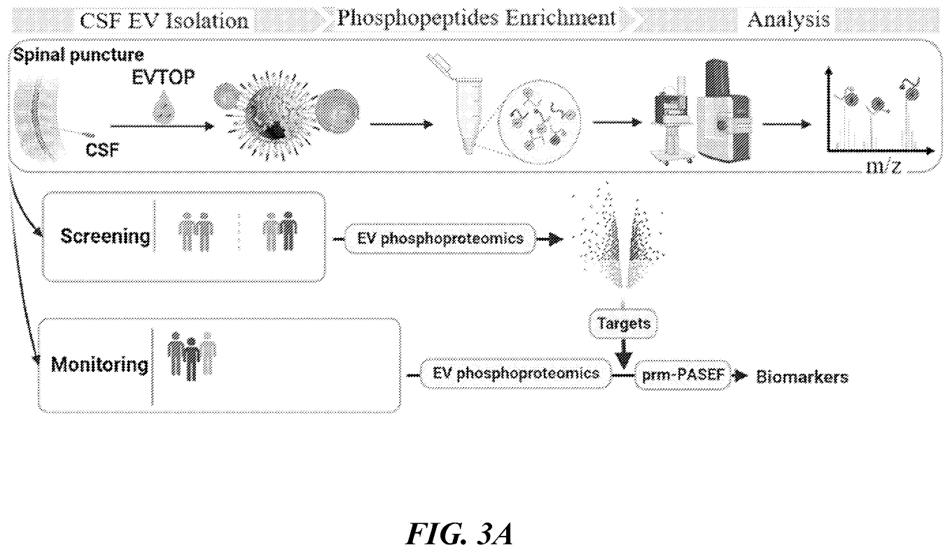

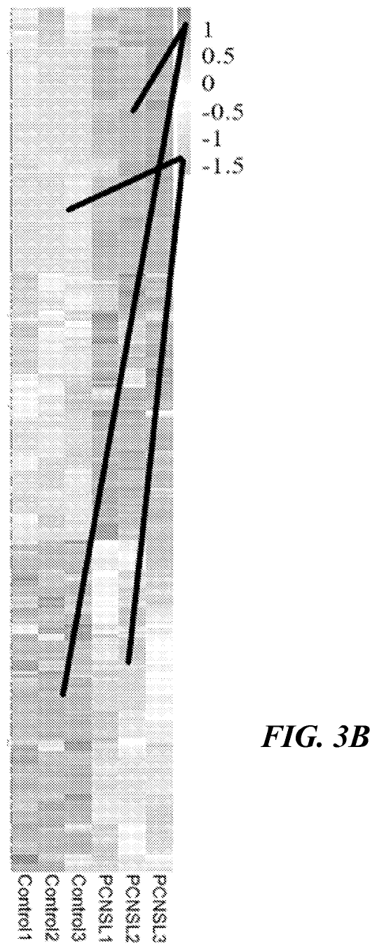

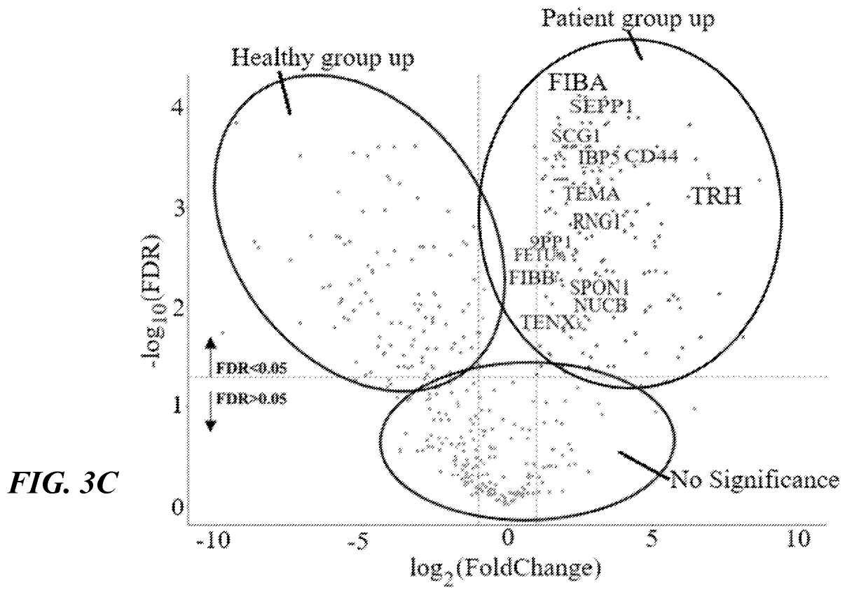

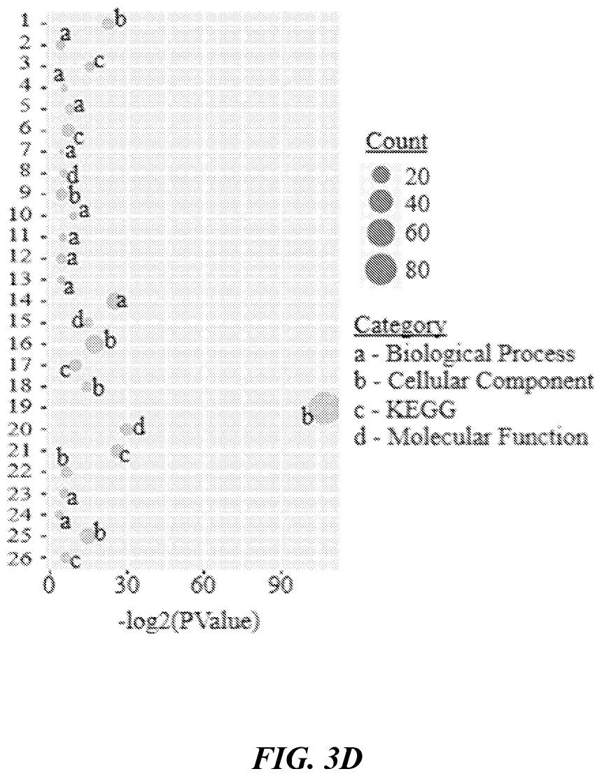

To demonstrate EVTOP's clinical applications, CSF samples collected from 21 PCNSL patients and 21 matched non-PCNSL control samples (collected and prepared as described above in Example 1) were analyzed for quantitative EV phosphoproteomics using the EVTOP beads and methods described herein (see FIG. 3A and Tables S9 and S10). More specifically, label-free quantitation was used, which employed software to normalize mass spectrometric signals followed by measuring peptide ion peak areas.

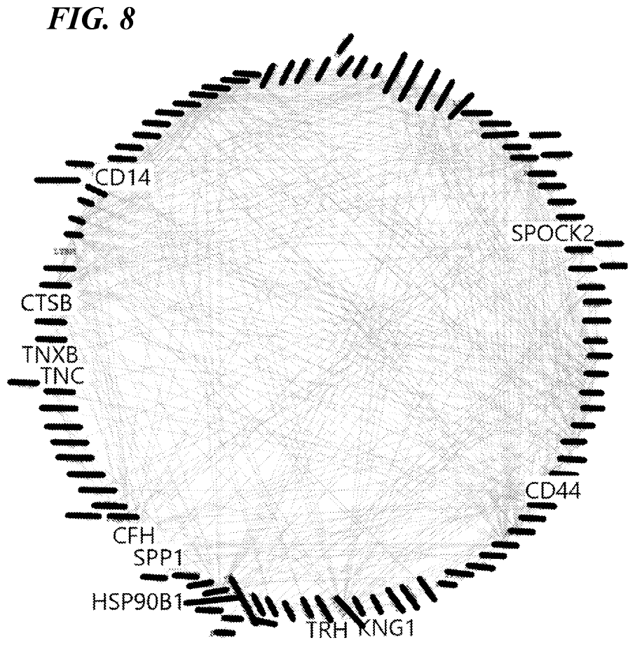

Collectively, 3,344 phosphopeptides representing 689 phosphoproteins were identified from CSF EVs, among which 160 phosphoproteins were upregulated in PCNSL patients (FIGS. 3B and 3C, and Table S11). Gene Ontology (GO), Kyoto Encyclopedia of Genes and Genomes (KEGG) pathway (FIG. 3D) and protein-protein interaction (PPI) analyses (FIG. 8) indicated these upregulated phosphoproteins were largely identified/predicted in extracellular exosome, cell surface, early endosome, and neuronal cell body, supporting their involvement in neuropeptide hormone activity, hyaluronic acid binding, and multiple signaling pathways such as Wnt signaling and PI3K-Akt pathways (see Table S12 showing a such results).

Several upregulated phosphoproteins previously reported as potential biomarkers for PCNSL and other cancers such as Osteopontin (SPP1), Cathepsin B (CTSB), Fibronectin (FN1), CD44, Tenascin-X (TNX), Myristoylated alanine-rich C-kinase substrate (MARCKS) and Testican-3 (SPOCK3) were identified.

### Example 7: Assessment of EV Phosphoproteins as Biomarkers for Treatment Efficacy

The feasibility of assessing the outcome of PCNSL chemotherapy using the identified EV phosphoproteins as potential biomarkers was also assessed.

An independent cohort of 11 PCNSL patients who were primarily treated with high dose of methotrexate and had overall good clinical response were selected for the study. 100 μL of CSF was collected from each patient both (a) before administration of chemotherapy, and (b) after administration of chemotherapy. The CSF samples were then subjected to the EVTOP method (using the EVTOP beads) to obtain EV phosphopeptides as described above. A combination of parallel reaction monitoring and parallel accumulation-serial fragmentation (prm-FASEF) on a timsTOF Pro instrument was then used to monitor the intensity changes of these upregulated phosphoproteins before and after chemotherapy. Lesur & Dittmar, The clinical potential of prm-PASEF mass spectrometry, Expert Review Proteomic 18(2): 75-82 (2021); Brzhozovskiy et al., The parallel reaction monitoring-parallel accumulation-serial fragmentation (prm-PASEF) approach for multiplexed absolute quantitation of proteins in human plasma, Analytical Chemistry 94(9): 2016-2022 (2022).

More specifically, the column used for LC-MS/MS analysis was home-packed with C18 resin (Michrom Bioresources Inc, Auburn, CA) with a particle size of 1.9 μm, wherein the inner diameter of the column was 75 microns and the length was 30 cm. The samples were dissolved in 0.1% formic acid solution prior to being subjected to a 60 minute gradient (gradient from 0 to 22% Solution B for 35 minutes, gradient from 22 to 37% Solution B for 15 minutes, gradient from 37 to 80% Solution B for 5 minutes, holding 80% Solution B for 5 minutes) with a flow rate of 300 nL/minute for phosphoproteomics analysis on timsTOF Pro (Bruker Corporation, Billerica, MA).

The MS analysis was carried out under data-dependent acquisition (DDA) mode conducted on full scan mode (from 100 to 1700 m/z, resolution at 40,000 and intensity of ‘target value’ more than 1.5×e⁶) with one full frame followed by 10 PASEF MS/MS frames in tandem. In addition, each of the PASEF MS/MS frames covered approximately 12 MS/MS spectra with ion mobility range of 0.6-1.6 Vs cm⁻²).

For the DDA data processing and bioinformatic analysis, the raw files search was conducted by PEAKS Studio X+ software (Bioinformatics Solutions Inc., Waterloo, Ontario, Canada) against the Uniprot database (obtained in December 2020) without repetitive entries. The tolerance of initial precursor was set to 15 ppm, final tolerance to 6 ppm, and MS/MS tolerance at 0.05 Da. The parameters for the peptide specimen search also included a static carbamidomethylation of cysteine (+57.02 Da) and variable modifications of oxidation (+15.99 Da) on methionine residues, acetylation (+42.01 Da) at the N terminus as well as phosphorylation (+79.97 Da) on serine, threonine, or tyrosine residues. The complete trypsin/P digestion was used for peptides searching against sequence database with a total of three missing cleavages allowed. A 1% (−10lgP≥20, ≥1 unique peptide for proteins) of false discovery rate (FDR) for peptides was set to remove the false matched peptides, and PEAKs Studio X+ was also employed to identify the localization of the phosphosites.

Prior to extracting the intensity of phosphopeptides, the initial precursor mass tolerance was set to 15 ppm, the minimum number of isotope peaks to 2, the maximum ART of isotope pattern multiples to −0.2 minutes, and propensity score matching (PSM) confidence FDR to 1%. The label-free quantitation (LFQ) method was used to compare phosphoproteomic differential expression according to the intensity of phosphoproteins calculated by those of phosphopeptides. The volcano plot and heatmap were generated using Perseus software according to the results of differential expression phosphoproteins identified by LFQ method conducted in the phosphoproteomics analysis of clinical samples (p-value <0.05, t test S₀=0, |log₂(Foldchange)|>1 were regarded as differential phosphoproteins) (see Table S11). 160 upregulated phosphoproteins were identified by the LFQ method.

The 160 upregulated phosphoproteins identified were then constructed into a phosphoprotein library. The healthy control pooled CSF sample, the PCNSL pooled CSF sample collected before administration of chemotherapy, and the PCNSL pooled CSF sample collected after administration of chemotherapy were analyzed in DDA mode (as described above) to select appropriate phosphopeptides as well as establish the coupling of the prm-PASEF method. By screening out the peptides that did not meet the conditions, 83 phosphopeptides were finally selected, corresponding to 51 phosphoproteins (see Table S14).

The monitored phosphopeptides were only kept within scans within +2.5 minutes of predicted retention time (RT) for each target. Resolution (60P) was selected as the type of ion mobility filter window. The spectrum library creation, peptides filter, and other prm-PASEF analysis were conducted by Skyline-daily (version 21.2.1.403). The selected phosphopeptides had a q-value of less than 1%, sequences of 8 to 25 amino acids were accepted, and the first 25 amino acids at the N-terminus of phosphoproteins were not included in the parent ion selection. The selected modifications were included to use in the prm-PASEF method establishment: Phospho (S, T) and Phospho (Y), Carboxymethyl (N-term), Oxidation, and Acetyl (N-term). Additionally, the prm-PASEF experiments were also performed on the pre-chemotherapy PCNSL-pooled CSF samples and post-chemotherapy PCNSL-pooled CSF samples to further refine the target list and test its feasibility before analyzing the individual CSF samples.

Following the analysis of individual CSF samples and the importation of prm-PASEF data, the same phosphopeptides were compared across runs and the adjustment of the RT location were carried out manually using Skyline-daily. The parent ion charge was +2 and +3, the product ion charge was +1, and the selected ion type contained b, y, p. The product ions from precursor to 5 product ions was set to pick the top 5 most intense product ions. All of the matched transitions were auto-selected.

The intensities of these phosphopeptides were extracted and the corresponding top 5 most intense product ions were summed, followed by the normalization of the total ion current (TIC) across runs. The intensity of each phosphoprotein was quantified using the sum of the intensities of all its including phosphopeptides, wherein the intensity of a phosphoprotein equates to the intensity summation of its corresponding phosphopeptides (see Table S15).

Phosphopeptides with relatively poor reproducibility, lower product ions coverage and that are understood to lack biological significance (those without known association with a disease or related biological pathway) were excluded, as were phosphopeptide isomers at the phosphorylation sites that did not result in good distinction with the analysis. Additionally, known high abundance proteins in plasma were also excluded in the selection process.

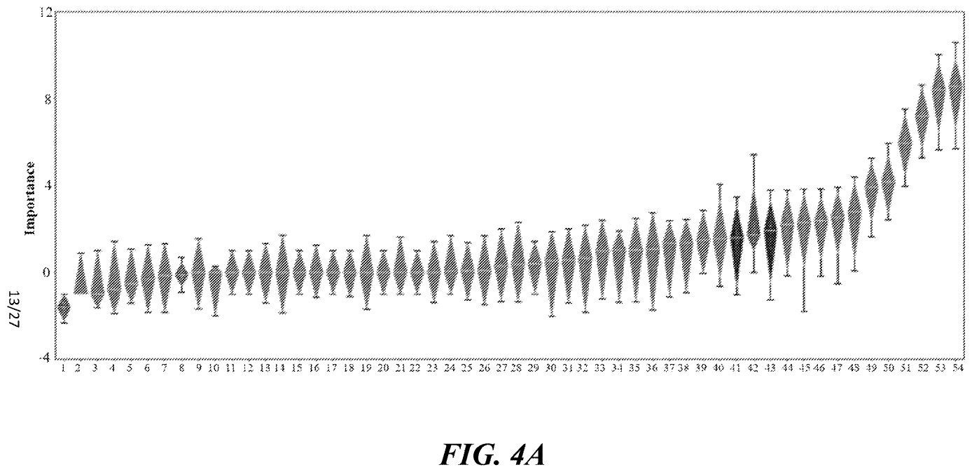

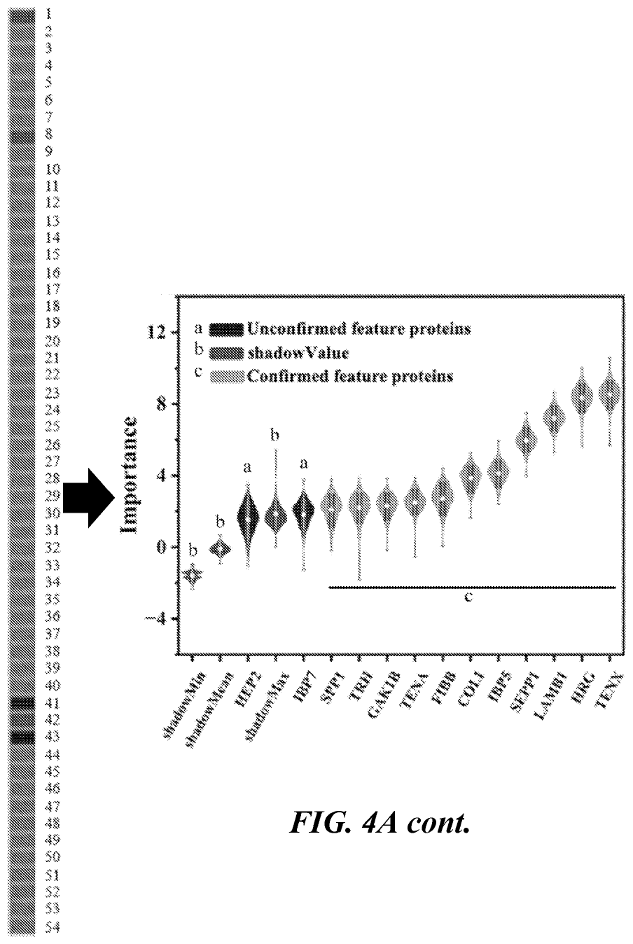

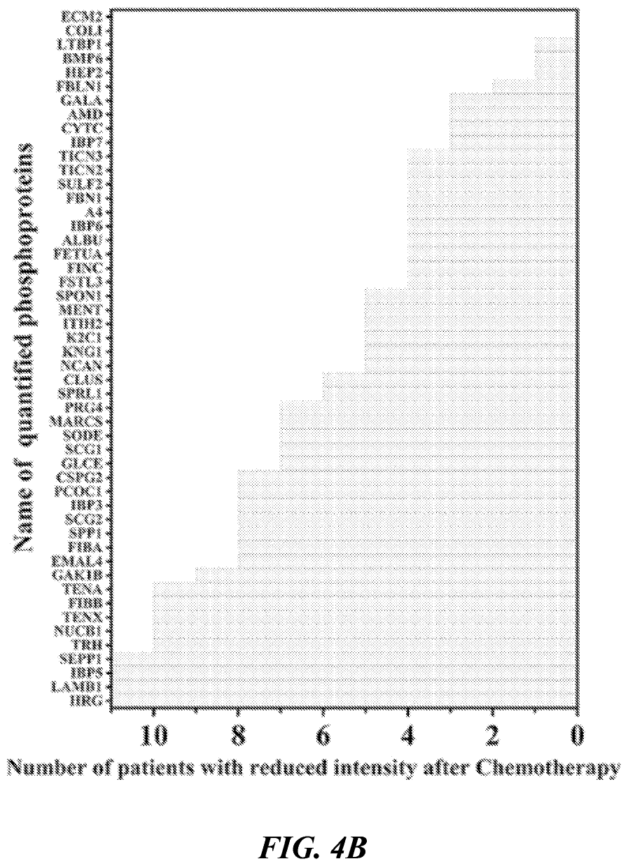

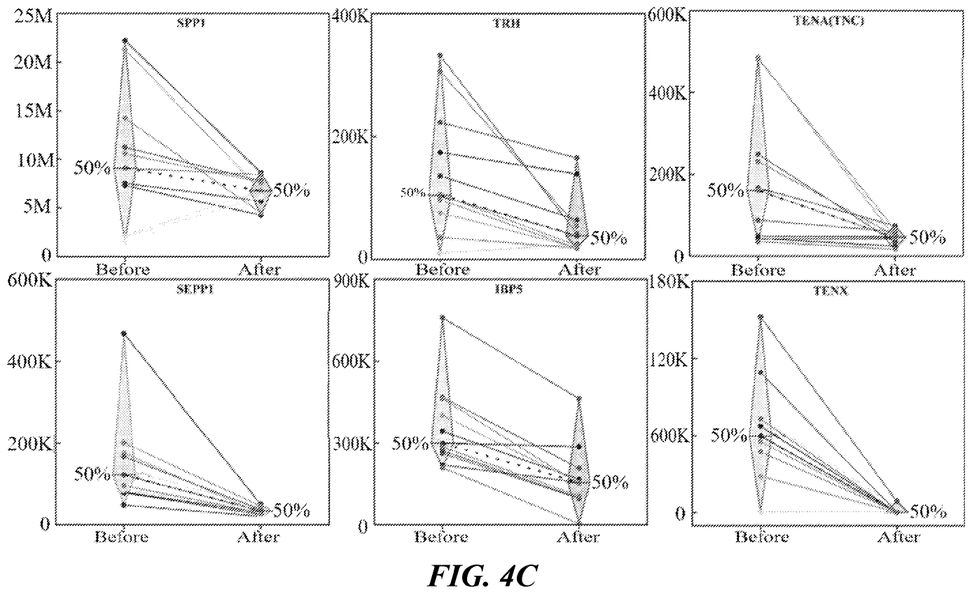

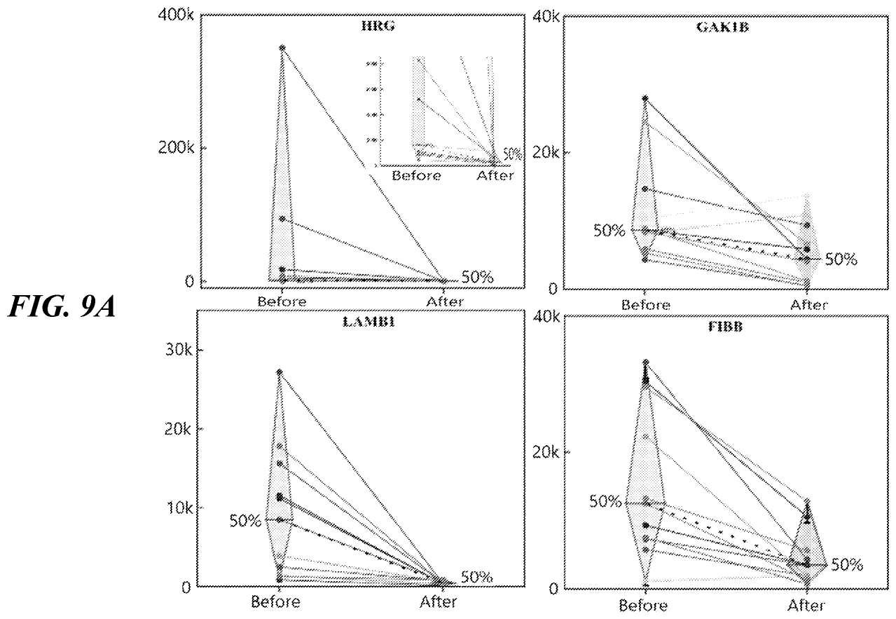

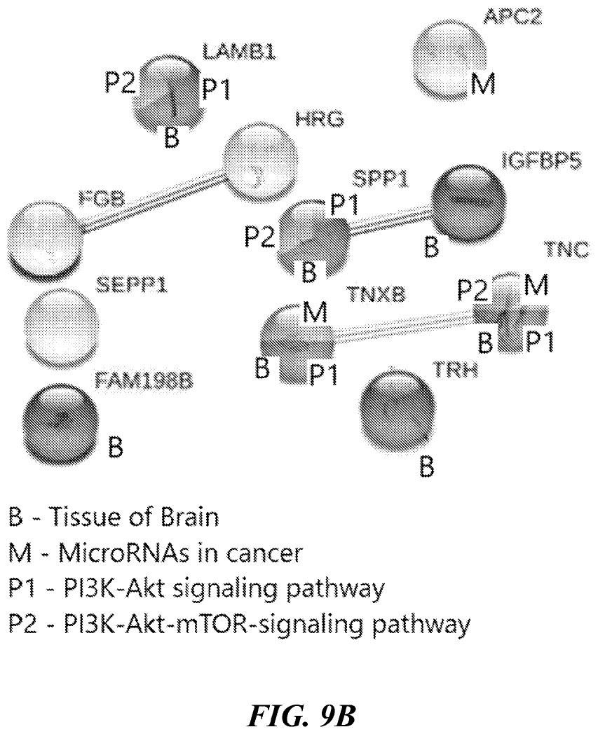

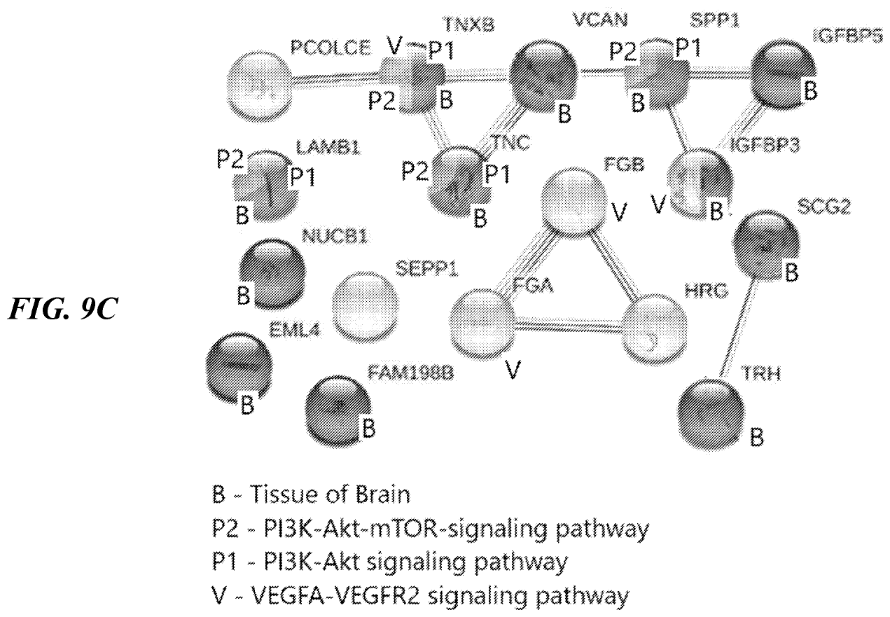

The resulting quantitative measurements of selected phosphopeptides are shown in the tables of Tables S13, S14, and S15. All quantified phosphoproteins selected in the prm-PASEF results were used to screen for the feature proteins (FIGS. 4A and 4B) and the following ten phosphoproteins showed distinctive signal decrease in 8 of the 11 patients after the chemotherapy equating with effective therapeutic intervention: SPP1, Pro-thyrotropin-releasing hormone (TRH), Tenascin-C(TNC), Selenoprotein P (SEPP1), Fibrinogen beta chain (FGB/FIBB), Laminin subunit beta-1 (LAMB1), TNX, Histidine-rich glycoprotein (HRG), Golgi-associated kinase 1B (GAK1B) and Insulin-like growth factor-binding protein 5 (IGFBP5/IBP5) (FIGS. 4C and 9A). Furthermore, the Reatcome/KEGG pathways analysis and PPI analysis indicated that 6 of the above-listed phosphoproteins belong to the brain tissue, and the majority of these biomarkers participate in PI3K-Akt, PI3K-Akt-mTOR, and other important signaling pathways, which have been reported to be related to the occurrence and development of PCNSL and act as therapeutic targets in treatment of various cancers and neurodegenerative diseases (FIGS. 9B and 9C). Takashima et al., Metabolome analysis reveals excessive glycolysis via PI3K/AKT/mTOR and RAS/MAPK signaling in methotrexate-resistant primary CNS lymphoma-derived cells, Clinical Cancer Research 26(11): 2754-2766 (2020); Duan & Yang, Therapeutic potential of PI3K/AKT/mTOR pathway in a gastrointestinal stromal tumors: rationale and progress, Cancers 12 (10): 2972 (2020). Similarly, phosphoproteins SPP1, TRH, TNC, SEPP1, IBP5, and TNX have been associated with neurological diseases, of which SPP1 is a reported biomarker for PCNSL screening. Pang et al., Osteopontin as a multifaceted driver of bone metastasis and drug resistance, Pharmacology Research 144:235-244 (2019); Tun et al., Pathway analysis of primary central nervous system lymphoma, Blood 111 (6): 3200-3210 (2008); Aho et al., Pathogenesis of primary central-nervous-system lymphoma—invasion of malignant lymphoid-cells into and within the brain parenchyme, Acta Neuropathology 86(1): 71-76 (1993); Sharma et al., CSF thyrotropin-releasing hormone concentrations differ in patients with schizoaffective disorder from patients with schizophrenia or mood disorders, J Psychiatric Research 35(5): 287-291 (2001); Tong et al., Silencing of Tenascin-C inhibited inflammation and apoptosis via PI3K/Akt/NF-kappa B signaling pathway in subarachnoid hemorrhage cell model, J Stroke & Cerebrovascular Diseases 29(1): 104485 (2020); Saito, Selenoprotein P as an in vivo redox regulator: disorders related to its deficiency and excess, J Clinical Biochemistry & Nutrition 66(1): 1-7 (2020); Tanno et al., Bim-dependent apoptosis follows IGFBP-5 down-regulation in neuroblastoma cells, Biochemistry & Biophysics Research Communications 351(2): 547-542 (2006); Kramer et al., Secretome identifies Tenascin-X as a potent marker of ovarian cancer, BioMed Research Int'l 2015: 208017 (2015); Cesi et al., Insulin-like growth factor binding protein 5: contribution to growth and differentiation of neuroblastoma cells, Annals NY Academy Science 1028:59-68 (2004); Sakai et al., A potential contribution of tenascin-X to blood vessel formation in peripheral nerves, Neuroscience Research 124: 1-7 (2017); Yuan et al., Tenascin-X is a novel diagnostic marker of malignant mesothelioma, Am J Surgical Pathology 33(11): 1673-1682 (2009).

These markers were reliably quantified from 100 uL of CSF before and after the administration of chemotherapy using the EVTOP beads and methods hereof, which highlights the power of the EVTOP technology hereof and its potential in future applications. Indeed, these results demonstrate the clinical application of EVTOP beads in assessing therapeutic outcome via profiling the phosphorylation landscape in EVs using a limited amount of CSF (or other biofluid) samples collected from patients with PCNSL.

1. A composition for recovery of phosphopeptides from cells and extracellular vesicles (EVs) comprising:
a solid phase support;
at least a cleavable membrane binding/penetrating group functionalizing a surface of the solid phase support, the cleavable membrane binding/penetrating group having an affinity for a cell or an EV; and
at least one metal ion or metal oxide functionalizing the surface of the solid support, the metal ion or metal oxide having an affinity for at least one phosphorylated residue in a phosphopeptide.

2. The composition of claim 1, wherein the metal ion is selected from the group consisting of iron, copper, gallium, cobalt, nickel, calcium, zinc, cadmium, silver, palladium, platinum, and ruthenium.

3. The composition of claim 1, wherein the metal ion is selected from the group consisting of titanium, zirconium, aluminum, vanadium, lead, manganese and tin.

4. The composition of claim 1, wherein the at least one metal ion or metal oxide comprises a Ti(IV) ion.

5. The composition of claim 1, wherein the at least one metal ion or metal oxide is positively charged.

6. The composition of claim 1, wherein the metal oxide is selected from the group consisting of titanium oxide, zirconium oxide and tin oxide, aluminum oxide, vanadium oxide, lead oxide and manganese oxide.

7. The composition of claim 1, wherein the solid phase support comprises a silica bead, magnetic bead, a polymer resin, or a combination of any two or more of the foregoing.

8. The composition of any one of claims 1-7, wherein the solid phase support comprises nanoparticles.

9. The composition of claim 1, wherein the solid phase support comprises nanoparticles.

10. The composition of any one of claims 1-7 and 9, further comprising a silica and/or a polymer shell coating at least a portion of the surface of the solid phase support.

11. The composition of claim 1, wherein the solid phase support further comprises polyethylene glycol and a silica shell coating at least a portion of the surface of the solid phase support.

12. The composition of claim 1, wherein a membrane binding/penetrating group can be cleaved by an enzyme, a chemical, by light, or by a combination of any two or more of the foregoing.

13. The composition of any one of claims 1-7 and 9, wherein the binding/penetrating group is selected from the group consisting of cell/membrane-penetrating peptides, membrane analogs, and membrane-bound molecules.

14. The composition of any one of claims 1-7 and 9, wherein the binding/penetrating group comprises at least octa-arginine R⁺₈.

15. The composition of claim 1, wherein the at least one metal ion or metal oxide comprises a Ti(IV) ion, and the binding/penetrating group comprises at least octa-arginine R⁺₈.

16. The composition of claim 1, wherein when subjected to an enzyme, a chemical, light, or a combination of any two or more of the foregoing, the binding/penetrating group is cleaved from the solid phase support while the at least one metal ion or metal oxide remains attached to the surface of the solid phase support.

17. The composition of any one of claims 1-7, 9 and 12, wherein the EVs comprise one or more of exosomes, small EVs (sEVs), ectosomes, microparticles, and microvesicles.

18. A kit for identifying phosphorylated peptides or the lack thereof, comprising:
a solid phase support;
at least a cleavable membrane binding/penetrating group functionalizing a surface of the solid phase support, the cleavable membrane binding/penetrating group having an affinity for a cell or an EV; and
at least one metal ion or metal oxide functionalizing the surface of the solid support, the metal ion or metal oxide having an affinity for at least one phosphorylated residue in a phosphopeptide.

19. The kit of claim 18, wherein the solid phase support comprises a silica bead, magnetic bead, a polymer resin, or a combination of any two or more of the foregoing.

20. The kit of claim 18, wherein the metal ion is selected from the group consisting of iron, copper, gallium, cobalt, nickel, calcium, zinc, cadmium, silver, palladium, platinum, and ruthenium.

21. The kit of claim 18, wherein the metal ion is selected from the group consisting of titanium, zirconium, aluminum, vanadium, lead, manganese and tin.

22. The kit of claim 18, wherein the at least one metal ion or metal oxide comprises a Ti(IV) ion.

23. The kit of claim 18, wherein the at least one metal ion or metal oxide is positively charged.

24. The kit of claim 18, wherein the metal oxide is selected from the group consisting of titanium oxide, zirconium oxide and tin oxide, aluminum oxide, vanadium oxide, lead oxide and manganese oxide.

25. The kit of any one of claims 18-24, wherein the solid phase support comprises nanoparticles.

26. The kit of claim 18, wherein the solid phase support comprises nanoparticles.

27. The kit of any one of claims 18-24 and 26, further comprising a silica and/or polymer shell coating at least a portion of the surface of the solid phase support.

28. The kit of claim 18, wherein the solid phase support further comprises polyethylene glycol and a silica shell coating at least a portion of the surface of the solid phase support.

29. The kit of claim 18, wherein a membrane binding/penetrating group can be cleaved by an enzyme, a chemical, by light, or by a combination of any two or more of the foregoing.

30. The kit of any one of claims 18-24 and 26, wherein the binding/penetrating group is selected from the group consisting of cell/membrane-penetrating peptides, membrane analogs, and membrane-bound molecules.

31. The kit of any one of claims 18-24 and 26, wherein the binding/penetrating group comprises at least octa-arginine R⁺₈.

32. The kit of claim 18, wherein the at least one metal ion or metal oxide comprises a Ti(IV) ion, and the binding/penetrating group comprises at least octa-arginine R⁺₈.

33. The kit of claim 18, wherein when subjected to an enzyme, a chemical, light, or a combination of any two or more of the foregoing, the binding/penetrating group is cleaved from the solid phase support while the at least one metal ion or metal oxide remains attached to the surface of the solid phase support.

34. The kit of any one of claims 18-24, 26, 32, and 33, wherein the EVs comprise one or more of exosomes, sEVs, ectosomes, microparticles, and microvesicles.

35. A method of one-pot capturing and enriching phosphoproteins, phosphopeptides, or both from a biofluid sample, the method comprising:
capturing EVs from a biofluid sample taken from a subject using a composition of any one of claims 1-17;
enriching the captured EVs by:
lysing the captured EVs while captured on the solid phase support,
digesting the captured EVs to generate phosphopeptides, and
cleaving the at least one membrane binding/penetrating group from the solid phase support.

36. The method of claim 35, wherein lysing the captured EVs is performed through application of a reagent in an amount sufficient to lyse the captured EV.

37. The method of claim 36, wherein the reagent comprises lithiumdodecyl sulfate.

38. The method of any one of claims 35-37, wherein digesting the captured EVs comprises digesting lysed proteins with Lys-C, trypsin, and/or other enzymes.

39. The method of claim 35, wherein digesting the captured EVs is performed for a time period and at a predefined temperature.

40. The method of claim 35, further comprising quantifying the phosphopeptides to determine if the subject is experiencing an upregulation, downregulation, or normal expression of the phosphopeptides as compared to a control sample, wherein upregulation or downregulation is indicative of the subject experiencing an active disease state and normal expression is indicative of the subject not experiencing the active disease state.

41. The method of claim 35, further comprising administering a treatment to the subject before or after collecting the biofluid sample from the subject.

42. The method of claim 41, wherein the treatment comprises chemotherapy.

43. The method of claim 41, further comprising quantifying the phosphopeptides to determine if the subject is experiencing an upregulation, downregulation, or normal expression of the phosphopeptides as compared to a control sample, wherein upregulation or downregulation is indicative of the subject experiencing an active disease state and normal expression is indicative of the subject not experiencing the active disease state.

44. The method of claim 40 or 42, wherein the disease state is cancer.

45. The method of claim 40 or 43, wherein the phosphopeptides comprise one or more of the following: SPP1, Pro-thyrotropin-releasing hormone (TRH), Tenascin-C (TNC), Selenoprotein P (SEPP1), Fibrinogen beta chain (FGB/FIBB), Laminin subunit beta-1 (LAMB1), TNX, Histidine-rich glycoprotein (HRG), Golgi-associated kinase 1B (GAK1B) and Insulin-like growth factor-binding protein 5 (IGFBP5/IBP5).

46. The method of claim 35, further comprising:
administering a treatment to the subject before collecting the biofluid sample from the subject;
quantifying the phosphopeptides to determine if the subject is experiencing an upregulation, downregulation, or normal expression of the phosphopeptides as compared to a control sample, wherein upregulation or downregulation is indicative of the subject experiencing an active disease state and normal expression is indicative of the subject not experiencing the active disease state; and
wherein the phosphopeptides comprise one or more of the following: SPP1, Pro-thyrotropin-releasing hormone (TRH), Tenascin-C(TNC), Selenoprotein P (SEPP1), Fibrinogen beta chain (FGB/FIBB), Laminin subunit beta-1 (LAMB1), TNX, Histidine-rich glycoprotein (HRG), Golgi-associated kinase 1B (GAK1B) and Insulin-like growth factor-binding protein 5 (IGFBP5/IBP5).

47. The method of claim 46, further comprising collecting a first biofluid sample from the subject prior administering the treatment and a second biofluid sample from the subject after administering the treatment; wherein downregulation of the phosphopeptides in the second biofluid sample as compared to the first fluid sample is indicative of the treatment effectively treating the disease state.

48. The method of any one of claims 35-47, wherein the biofluid sample is plasma, urine, saliva, or cerebral spinal fluid (CSF).

49. The method of any one of claims 35-48, wherein the biofluid sample is CSF.

50. The method of any one of claims 35-49, wherein the biofluid sample comprises between about 10 μL to about 200 μL, inclusive of the end points.

51. The method of any one of claims 35-48 and 50, wherein the biofluid sample comprises about 10 μL of plasma.

52. The method of any one of claims 35-50, wherein the biofluid sample comprises about 100 μL of CSF.

53. The method of any one of claims 35-48 and 50, wherein the biofluid sample comprises about 200 μL of urine.

54. The method of any one of claims 35-48 and 50, wherein the biofluid sample comprises about 50 μL of saliva.

55. Use of the composition of any one of claims 1-17 for assessing a physiological response in a subject, or lack thereof, to an administered treatment.

56. The use of claim 55, wherein the treatment is chemotherapy.
